(19) United States
(12) Patent Application Publication
(10) Pub. No.: US 2004/0152667 A1
(43) Pub. Date: Aug. 5, 2004

(54) 4-Alkenylthiazoles comprising epoxide functionality, and methods of use thereof

(76) Inventors: James R. Hauske, Concord, MA (US); Joanne M. Holland, Brookline, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/690,478

(22) Filed: Oct. 21, 2003

Related U.S. Application Data
(60) Provisional application No. 60/420,674, filed on Oct. 23, 2002.

(51) Int. Cl. A61K 31/695; A61K 31/427; C07F 7/02; C07D 417/02

(52) U.S. Cl. 514/63; 514/365; 514/151; 548/110; 548/203

(57) ABSTRACT

One aspect of the present invention relates to heterocyclic compounds. A second aspect of the present invention relates to the use of the heterocyclic compounds as ligands for various mammalian cellular receptors, including G-protein-coupled receptors (GPCRs). A third aspect of the present invention relates to the use of the heterocyclic compounds as ligands for various mammalian kinases. The compounds of the present invention will also find use in the treatment of numerous ailments, conditions and diseases which afflict mammals, including but not limited to addiction, anxiety, depression, sexual dysfunction, hypertension, migraine, Alzheimer's disease, obesity, emesis, psychosis, analgesia, schizophrenia, Parkinson's disease, restless leg syndrome, sleeping disorders, attention deficit hyperactivity disorder, irritable bowel syndrome, premature ejaculation, menstrual dysphoria syndrome, urinary incontinence, inflammatory pain, neuropathic pain, Lesche-Nyhane disease, Wilson's disease, Tourette's syndrome, psychiatric disorders, stroke, senile dementia, peptic ulcers, pulmonary obstruction disorders, asthma, cancer, cell proliferative disorders, fibrotic disorders, metabolic disorders, and diabetes. The present invention also relates to combinatorial libraries of the novel compounds, and methods of preparing said libraries.

### RELATED APPLICATIONS

This application claims the benefit of priority to U.S. Provisional Patent Application serial No. 60/420,674, filed Oct. 23, 2002.

### BACKGROUND OF THE INVENTION

G-Protein-Coupled Receptors (GPCRs)

There are trillions of cells in the normal, healthy human body, and how these cells communicate is critical to their physiological behavior. When cell-to-cell communication is unsuccessful or inappropriate, the result can be a harmful behavior or response, such as cancer. Cells have hundreds of receptors on their surface. These receptors serve as the antenna for cells—they catch molecules such as hormones and neurotransmitters and relay information to the inside of the cell. Reception of a signal involves attachment of the signal, via a receptor, to the recipient cell.

G-Protein Coupled Receptors (GPCRs), or 7-transmembrane receptors, all have a structure that is characterized by 7 domains that cross the cell's membrane and are often called serpentine receptors. These serpentine receptors are of ancient lineage, going back as far as yeast. A well-known example of a GPCR is the beta adrenergic receptor, which relays the epinephrine/adrenaline signal. The result of the signal relay by this receptor is the establishment of the primitive mammalian fight- or -flight response. Other common examples of GPCRs include visual receptors, odor receptors, taste receptors, hormone receptors (e.g. glucagon, angiotensin, vasopressin), and the HIV co-receptor (CCR5). Further, individual ligands for GPCRs have been shown to be active as antihistamines (allergies), bronchodilators (asthma), beta blockers (high blood pressure), opioids (pain), or migraine drugs.

Psychiatric disorders are pathological conditions of the brain characterized by identifiable symptoms that results in abnormalities in cognition, emotion or mood, or the highest integrative aspects of behavior. These disorders may vary in severity of symptoms, duration, and functional impairment. Psychiatric disorders afflict millions of people worldwide resulting in tremendous human suffering and economic burden due to lost productivity.

Psychiatric disorders can be classified into various categories based on etiology and symptomatology. Such a classification system includes somatoform disorders, anxiety disorders, dissociative disorders, mood disorders, personality disorders, psychosexual disorders, schizophrenia and related disorders, drug abuse and dependence, and eating disorders.

The pathophysiological mechanisms responsible for psychiatric disorders are very complex. However, with increasing understanding of neuroanatomy and neurophysiology these mechanisms and the effect of pharmacological agents on these mechanisms is becoming clearer. Protein molecular targets that psychopharmaceuticals interact with to have an effect can be divided into three general classes: (1) enzymes; (2) ion channels; and (3) G-protein coupled receptors (GPCR's). The current molecular targets believed to be involved in the pathology of psychiatric disorders predominately are GPCRs. Consequently, many of the current psychotherapeutics used today are ligands for GPCRs.

Despite the many advances that occurred from a better understanding of neuropharmacology, many psychiatric diseases remain untreated or inadequately treated with current pharmaceutical agents. In addition, many of the current agents interact with molecular targets not involved with the psychiatric disease. This indiscriminate binding can result in side effects that can greatly influence the overall outcome of therapy. In some cases the side effects are so severe that discontinuation of therapy is required. For example, research into the development of new, selective ligands for neuronal GPCRs holds the promise of yielding potent compounds for the treatment of psychiatric disorders that lack the side effects of current therapies.

Protein Kinases

Protein kinases represent the largest superfamily of homologous proteins with over 300 mammalian members known to date and a greater number predicted from genome sequencing and analysis. Despite their involvement in numerous and diverse cellular pathways, the majority of protein kinases share several common sequence and structural motifs. Tertiary structure determination has revealed that the highly conserved region of 250-300 amino acids delineated by sequence similarity, folds into a common catalytic core. The bi-lobal structure formed consists of 13 conserved subdomains allowing kinases to perform their three main roles, namely, binding and orientation of ATP (complexed with a divalent cation) binding and orientation of substrate and, thirdly, phosphate transfer. The smaller N-terminal lobe consists predominantly of anti-parallel beta sheets and is involved in the anchoring of the nucleotide. The larger C-terminal lobe is alpha helical, binds the substrate and initiates transfer of phosphate. The cleft between the lobes is the site of catalysis.

The high degree of relatedness between members of the superfamily has allowed phylogenetic relationships to be examined. In mammalian kinases, two main subdivisions exist based on substrate specificity; Protein Tyrosine Kinases (PTKs) and Serine/Threonine Protein Kinases (S/T Ks) with short amino acid stretches characterizing each class. Each class can be further divided into sub-classes and subfamilies. Tyrosine Kinases consist of both receptor tyrosine kinases (RTKs) and cytosolic kinases with several families within each group. Three main groups occur in the S/T K family. The AGC group includes the cyclic nucleotide-regulated protein kinase families (PKA and PKG) and the diacylglycerol activated/phospholipid dependent (PKC) family and related kinases. The CaMK group of kinases contains the family of kinases regulated by Ca²⁺/Calmodulin and related subfamilies. The CMGC group contains numerous families including the cyclin dependent kinases (CDK), the MAPK/Erk family as well as the glycogen synthase (GSK-3) and the Clk families. In addition, there are several kinase families that do not fall within these main groups.

Eukaryotic protein kinases constitute a large family of homologous proteins that catalyze the transfer of a phosphate group of ATP or GTP to the hydroxyl group of serine, threonine or tyrosine in a substrate protein. Protein kinases differ in their structure, subcellular location, substrate specificity, and function. Cellular signaling cascades rely on the phosphorylation status of proteins in their pathways. Phosphorylation cascades integrate and regulate many cellular processes. Additionally, phosphorylation allows for protein-protein interaction which results in enzyme activation. Phosphorylated proteins are substrates for specific protein phosphatases so that phosphorylation and dephosphorylation serve as molecular switches.

Gene expression, cytoskeletal integrity, cell adhesion, cell cycle progression, and differentiation are controlled by the complex interplay of protein kinases and phosphatases in specific signaling pathways. Malfunctions of cellular signaling have been linked with many diseases including cancer and diabetes. Regulation of signal transduction pathways by cytokines and the association of signal molecules with protooncogenes and tumor suppressor genes have been subjects of intense research leading to new therapeutic possibilities. In a multicellular organism, intercellular communication plays a crucial role under normal as well as pathological conditions. Normal cells provide the stroma and blood supply essential for maintaining growth and progression of tumors. Such codependence relies on a wide array of receptors and signal transduction pathways of either the host or cancer cell. Mutant tyrosine kinases are also often associated with the development and progression of cancer, making tyrosine kinase signaling pathways attractive targets for oncology research. Receptor tyrosine kinases have been shown to be involved in signaling by and among tumor cells and host tissues.

Nearly a third of all the proteins inside of cells are phosphorylated by protein kinases. In many cases, this leads to direct regulation of these proteins through local changes in their structures. Some proteins are enzymes that have their catalytic activities rapidly turned on by their phosphorylation. Conversely, different enzymes are quickly turned off upon their phosphorylation. Most phosphoproteins are phosphorylated at multiple sites by distinct protein kinases. This permits integration of multiple signalling pathways into communication networks or webs.

The normal cell cycle involves a precise order of events, culminating in cell growth and division. With diseases such as cancer, the cell division cycle becomes deregulated, allowing tumor cells to proliferate in an uncontrolled manner. Tyrosine kinases and serine-threonine kinases and their signaling pathways control the growth, differentiation and programmed death of cells in response to extracellular signals such as hormones and growth factors. Alterations in these signaling pathways could promote cancer by encouraging uncontrolled and abnormal cell growth.

Approximately fifty genes that have been directly linked to induction of cancer (i.e. oncogenes) encode protein kinases. Many of the remaining oncogenes specify proteins that either activate kinases or are phosphorylated by kinases. Although the findings are less direct, aberrant cell signalling through protein kinases has also been associated with cardiovascular disease, diabetes, inflammation, arthritis and other immune disorders, and neurological disorders, such as Alzheimer's disease. Over 400 human diseases have been connected to protein kinases.

### SUMMARY OF THE INVENTION

One aspect of the present invention relates to heterocyclic compounds. A second aspect of the present invention relates to the use of the heterocyclic compounds as ligands for various mammalian cellular receptors, including G-protein-coupled receptors (GPCRs). A third aspect of the present invention relates to the use of the heterocyclic compounds as ligands for various mammalian kinases. The compounds of the present invention will also find use in the treatment of numerous ailments, conditions and diseases which afflict mammals, including but not limited to addiction, anxiety, depression, sexual dysfunction, hypertension, migraine, Alzheimer's disease, obesity, emesis, psychosis, analgesia, schizophrenia, Parkinson's disease, restless leg syndrome, sleeping disorders, attention deficit hyperactivity disorder, irritable bowel syndrome, premature ejaculation, menstrual dysphoria syndrome, urinary incontinence, inflammatory pain, neuropathic pain, Lesche-Nyhane disease, Wilson's disease, Tourette's syndrome, psychiatric disorders, stroke, senile dementia, peptic ulcers, pulmonary obstruction disorders, asthma, cancer, cell proliferative disorders, fibrotic disorders, metabolic disorders, and diabetes. The present invention also relates to combinatorial libraries of the novel compounds, and methods of preparing said libraries.

### BRIEF DESCRIPTION OF THE FIGURES

FIG. 1 depicts a retrosynthetic analysis.

FIG. 2 depicts a retrosynthetic analysis of an α-aminoepoxide.

### DETAILED DESCRIPTION OF THE INVENTION

Individual compounds described herein promise to have agonistic, antagonistic, and hybrid effects on GPCRs or protein kinases. One aspect of the present invention relates to the use of compounds of the present invention to treat diseases, afflictions, or maladies caused, at least in part, by abnormal activity of one or more GPCRs or protein kinase. Additionally, the compounds of the present invention will also find use in the treatment of numerous ailments, conditions and diseases which afflict mammals, including but not limited to addiction, anxiety, depression, sexual dysfunction, hypertension, migraine, Alzheimer's disease, obesity, emesis, psychosis, analgesia, schizophrenia, Parkinson's disease, restless leg syndrome, sleeping disorders, attention deficit hyperactivity disorder, irritable bowel syndrome, premature ejaculation, menstrual dysphoria syndrome, urinary incontinence, inflammatory pain, neuropathic pain, Lesche-Nyhane disease, Wilson's disease, Tourette's syndrome, psychiatric disorders, stroke, senile dementia, peptic ulcers, pulmonary obstruction disorders, asthma, cancer, cell proliferative disorders, fibrotic disorders, metabolic disorders, and diabetes. Further, compounds reported herein may possess properties for treating psychiatric disorders and other neurological conditions free of side effects encountered with currently available therapies.

Definitions

For convenience, certain terms employed in the specification, examples, and appended claims are collected here.

The term “cancer” refers to various types of malignant neoplasms, most of which can invade surrounding tissues, and may metastasize to different sites, as defined by Stedman's Medical Dictionary 25th edition (Hensyl ed. 1990). Examples of cancers which may be treated by the present invention include, but are not limited to, leukemia, and pancreatic, gastric, brain, ovarian, colon, prostate, kidney, bladder, breast, lung, oral and skin cancers which exhibit inappropriate protein kinase activity. Further, brain cancers include glioblastoma multiforme, anaplastic astrocytoma, astrocytoma, ependymoma, oligodendroglioma, medulloblastoma, meningioma, sarcoma, hemangioblastoma, and pineal parenchymal. Skin cancers include melanoma and Kaposi's sarcoma.

The term “cell surface proteins” includes molecules that occur on the surface of cells, interact with the extracellular environment, and transmit or transduce information regarding the environment intracellularly.

The term “extracellular signals” includes a molecule or a change in the environment that is transduced intracellularly via cell surface proteins that interact, directly or indirectly, with the signal. An extracellular signal is any compound or substance that in some manner specifically alters the activity of a cell surface protein. Examples of such signals include, but are not limited to, molecules such as acetylcholine, growth factors, hormones and other mitogenic substances, such as phorbol mistric acetate (PMA), that bind to cell surface receptors and ion channels and modulate the activity of such receptors and channels. Extracellular signals also includes as yet unidentified substances that modulate the activity of a cell surface protein and thereby affect intracellular functions and that are potential pharmacological agents that may be used to treat specific diseases by modulating the activity of specific cell surface receptors.

The term “ED₅₀” means the dose of a drug which produces 50% of its maximum response or effect. Alternatively, the dose which produces a pre-determined response in 50% of test subjects or preparations.

The term “LD₅₀” means the dose of a drug which is lethal in 50% of test subjects.

The term “therapeutic index” refers to the therapeutic index of a drug defined as LD₅₀/ED₅₀.

The term “structure-activity relationship (SAR)” refers to the way in which altering the molecular structure of drugs alters their interaction with a receptor, enzyme, etc.

The term “agonist” refers to a compound that mimics the action of natural transmitter or, when the natural transmitter is not known, causes changes at the receptor complex in the absence of other receptor ligands.

The term “antagonist” refers to a compound that binds to a receptor site, but does not cause any physiological changes unless another receptor ligand is present.

The term “competitive antagonist” refers to a compound that binds to a receptor site; its effects can be overcome by increased concentration of the agonist.

The term “partial agonist” refers to a compound that binds to a receptor site but does not produce the maximal effect regardless of its concentration.

The term “inverse agonist” refers to a compound that binds to a constitutively active receptor site and reduces its physiological function.

The term “ligand” refers to a compound that binds at the receptor site.

The term “heteroatom” as used herein means an atom of any element other than carbon or hydrogen. Preferred heteroatoms are boron, nitrogen, oxygen, phosphorus, sulfur and selenium.

The term “electron-withdrawing group” is recognized in the art, and denotes the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, J. March, Advanced Organic Chemistry, McGraw Hill Book Company, New York, (1977 edition) pp. 251-259. The Hammett constant values are generally negative for electron donating groups (σ[P]=−0.66 for NH₂) and positive for electron withdrawing groups (σ[P]=0.78 for a nitro group), σ[P] indicating para substitution. Exemplary electron-withdrawing groups include nitro, acyl, formyl, alkylsulfonyl, arylsulfonyl, trifluoromethyl, cyano, chloride, and the like. Exemplary electron-donating groups include amino, methoxy, and the like.

The term “alkyl” refers to the radical of saturated a liphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, alkylsulfonyl, arylsulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, alkylsulfonyl, arylsulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, alkylsulfonyl, arylsulfonyl, ketone, aldehyde, ester, a heterocyclyl, an aromatic or heteroaromatic moiety, —CF₃, —CN, or the like.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formula:

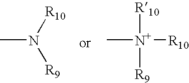

wherein R₉, R₁₀ and R′₁₀ each independently represent a group permitted by the rules of valence.

The term “acylamino” is art-recognized and refers to a moiety that can be represented by the general formula:

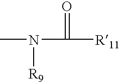

wherein R₉ is as defined above, and R′₁₁ represents a hydrogen, an alkyl, an alkenyl or —(CH₂)ₘ—R₈, where m and R₈ are as defined above.

The term “amido” is art recognized as an amino-substituted carbonyl and includes a moiety that can be represented by the general formula:

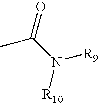

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S—alkyl, —S-alkenyl, —S-alkynyl, and —S—(CH₂)ₘ—R₈, wherein m and R₈ are defined above. Representative alkylthio groups include methylthio, ethyl thio, and the like.

The term “carbonyl” is art recognized and includes such moieties as can be represented by the general formula:

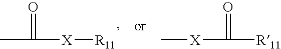

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R′₁₁ is as defined above, the moiety is referred to herein as a carboxyl group, and particularly when R₁₁ is a hydrogen, the formula represents a “carboxylic acid”. Where X is an oxygen, and R₁₁ is hydrogen, the formula represents a “formate”. In general, where the oxygen atom of the above formula is replaced by sulfur, the formula represents a “thiol carbonyl” group. Where X is a sulfur and R₁₁ or R′₁₁ is not hydrogen, the formula represents a “thiolester.” Where X is a sulfur and R₁₁ is hydrogen, the formula represents a “thiol carboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “sulfonate” is art recognized and includes a moiety that can be represented by the general formula:

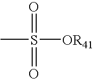

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is art recognized and includes a moiety that can be represented by the general formula:

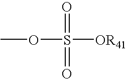

in which R₄₁ is as defined above.

The term “sulfonylamino” is art recognized and includes a moiety that can be represented by the general formula:

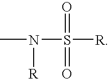

The term “sufamoyl”is art-recognized and includes a moiety that can be represented by the general formula:

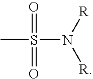

The term “sulfonyl”, as used herein, refers to a moiety that can be represented by the general formula:

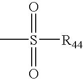

in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula:

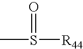

in which R₄₄ is selected from the group consisting of hydrogen, alkyl, alkenyl, alkynyl, cycloalkyl, heterocyclyl, aralkyl, or aryl.

A “selenoalkyl” refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R₇, m and R₇ being defined above.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substituation is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., functioning as analgesics), wherein one or more simple variations of substituents are made which do not adversely affect the efficacy of the compound in binding to sigma receptors. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

Cell Surface Receptors

Cell surface receptors and ion channels are among the cell surface proteins that respond to extracellular signals and initiate the events that lead to this varied gene expression and response. Ion channels and cell surface-localized receptors are ubiquitous and physiologically important cell surface membrane proteins. They play a central role in regulating intracellular levels of various ions and chemicals, many of which are important for cell viability and function.

Cell surface-localized receptors are membrane spanning proteins that bind extracellular signalling molecules or changes in the extracellular environment and transmit the signal via signal transduction pathways to effect a cellular response. Cell surface receptors bind circulating signal polypeptides, such as neurotransmitters, growth factors and hormones, as the initiating step in the induction of numerous intracellular pathways. Receptors are classified on the basis of the particular type of pathway that is induced. Included among these classes of receptors are those that bind growth factors and have intrinsic tyrosine kinase activity, such as the heparin binding growth factor (HBGF) receptors, and those that couple to effector proteins through guanine nucleotide binding regulatory proteins, which are referred to as G protein coupled receptors and G proteins, respectively.

The G protein transmembrane signaling pathways consist of three proteins: receptors, G proteins and effectors. G proteins, which are the intermediaries in transmembrane signaling pathways, are heterodimers and consist of alpha, beta and gamma subunits. Among the members of a family of G proteins the alpha subunits differ. Functions of G proteins are regulated by the cyclic association of GTP with the alpha subunit followed by hydrolysis of GTP to GDP and dissociation of GDP.

G protein coupled receptors are a diverse class of receptors that mediate signal transduction by binding to G proteins. Signal transduction is initiated via ligand binding to the cell membrane receptor, which stimulates binding of the receptor to the G protein. The receptor G protein interaction releases GDP, which is specifically bound to the G protein, and permits the binding of GTP, which activates the G protein. Activated G protein dissociates from the receptor and activates the effector protein, which regulates the intracellular levels of specific second messengers. Examples of such effector proteins include adenyl cyclase, guanyl cyclase, phospholipase C, and others.

G protein-coupled receptors, which are glycoproteins, are known to share certain structural similarities and homologies (see, e-g., Gilman, A. G., Ann. Rev. Biochem.56: 615-649 (1987), Strader, C. D. et al. The FASEB Journal 3: 1825-1832 (1989), Kobilka, B. K., et al. Nature 329:75-79 (1985) and Young et al. Cell 45: 711-719 (1986)). Among the G protein-coupled receptors that have been identified and cloned are the substance P receptor, the angiotensin receptor, the alpha- and beta-adrenergic receptors and the serotonin receptors. G protein-coupled receptors share a conserved structural motif. The general and common structural features of the G protein-coupled receptors are the existence of seven hydrophobic stretches of about 20-25 amino acids each surrounded by eight hydrophilic regions of variable length. It has been postulated that each of the seven hydrophobic regions forms a transmembrane alpha helix and the intervening hydrophilic regions form alternately intracellularly and extracellularly exposed loops. The third cytosolic loop between transmembrane domains five and six is the intracellular domain responsible for the interaction with G proteins.

G protein-coupled receptors are known to be inducible. This inducibility was originally described in lower eukaryotes. For example, the cAMP receptor of the cellular slime mold, Dictyostelium, is induced during differentiation (Klein et al., Science 241: 1467-1472 (1988). During the Dictyostelium discoideum differentiation pathway, cAMP, induces high level expression of its G protein-coupled receptor. This receptor transduces the signal to induce the expression of the other genes involved in chemotaxis, which permits multicellular aggregates to align, organize and form stalks (see, Firtel, R. A., et al. Cell 58: 235-239 (1989) and Devreotes, P., Science 245: 1054-1058 (1989)).

Protein Kinases

Protein kinase catalyzed phosphorylation of the hydroxyl moiety of serine, threonine or tyrosine is the central post-translational control element in eukaryotic signal transduction. The phosphorylation state of a given protein can govern its enzyme activity, protein-protein binding interactions, and cellular distribution. Phosphorylation and dephosphorylation is thus a “chemical switch” which allows the cell to transmit signals from the plasma membrane to the nucleus to ultimately control gene expression in a highly regulated manner.

Protein kinase B (PKB/Akt) is a component of an intracellular signalling pathway of fundamental importance that functions to exert the effects of growth and survival factors, and which mediates the response to insulin and inflammatory signals. The enzyme is rapidly activated by phosphorylation following stimulation of phosphoinositide 3-kinase, and generation of the lipid second messenger phosphatidylinositol 3,4,5 trisphosphate [PtdIns(3,4,5)P3]. Activation of PKB occurs by a multi-step mechanism. PKB is first recruited to the membrane by association with PtdIns(3,4,5)P3 mediated by its N-terminal pleckstrin homology domain in a process that also induces a conformational change of the protein. In this state, PKB is a substrate for phosphorylation at two regulatory sites by membrane-localised kinases. PDK1 phosphorylates PKB on a Thr residue (Thr-309 of PKBβ) within the activation segment, whereas a distinct kinase activity, termed PDK2, phosphorylates PKB at Ser-474 of its C-terminal hydrophobic motif. Activated PKB phosphorylates numerous proteins, regulating diverse cellular processes.

Raf kinase is a proto-oncogene that links activated cell surface receptors to the ERK1/2 by MEK phosphorylation. The activity of Raf-kinase is subject to intricate regulatory mechanisms, that includes control mediated by Ras, reversible Ser/Thr and Tyr phosphorylation, and 14-3-3 interactions. Furthermore, Hsp90 and p50[CDC37] are important for maintaining Raf activity. Current models for Raf regulation suggest that differential phosphorylation and 14-3-3 association allows for intrasteric inhibition of the C-terminal active kinase domain by the conserved N-terminal regulatory domain of Raf-1. Membrane localised interactions with these domains and Ras-GTP with further phosphorylation immediately N-terminal to the protein kinase domain alleviates inhibition and triggers partial Raf kinase activity. Full activity is achieved in concert with activated Src. B-Raf activation differs from Raf-1 in that the former has a higher level of constitutive phosphorylation and the presence of Asp residues equivalent to phospho-Tyr residues of Raf-1.

Protein kinase C (also known as “calcium/phospholipid-dependent protein kinase”, “PKC” or “C-kinase) is a family of very closely related enzymes; one or more members of the protein kinase C family are found in nearly all animal tissues and animal cells that have been examined. The identity of protein kinase C is generally established by its ability to phosphorylate certain proteins when adenosine triphosphate and phospholipid cofactors are present, with greatly reduced activity when these cofactors are absent. Protein kinase C is believed to phosphorylate only serine and/or threonine residues in the proteins that are substrates for protein kinase C.

It is well established that PKC family proteins play central roles in cell growth and differentiation. PKCs mediate the effects of peptide hormones, growth factors, neurotransmitters and tumor promoters by acting as secondary (downstream, intracellular) messengers for these signaling molecules (Y. Nishizuka, Science 233, 305-312 (1986); Y. Takai, K. Kaibuchi, T. Tsuda, M. Hoshijima, J. Cell. Biochem. 29, 143-155 (1985)). The identities of the PKC isozymes that transduce particular signals in specific cell types are still being determined. The alpha., .beta.I, .beta.II, gamma., .delta., .epsilon. and .zeta. isozymes have been implicated in the differentiation of normeural cells (E. Berra, et al., Cell 74, 555-563 (1993); J. Goodnight, H. Mischak, J. F. Mushinski, Adv. Cancer Res. 64, 159-209 (1994); J. R. Gruber, S. Ohno, R. M. Niles, J. Biol. Chem. 267, 13356-13360 (1992); D. E. Macfarlane, L. Manzel, J. Biol. Chem. 269, 4327-4331 (1994); C. T. Powell et al., Proc. Natl. Acad. Sci. USA 89, 147-151 (1992)). Recent studies, showing that the .epsilon. isozyme of PKC (“PKC.epsilon.”) is activated by nerve growth factor (“NGF”) and mediates NGF-induced neurite outgrowth, were interpreted as indicating a role for PKC.epsilon. in neuronal differentiation (B. Hundle, et al., J. Biol. Chem. 272, 15028-15035 (1997)).

Some forms of protein kinase C require the presence of calcium ions for maximal activity. Protein kinase C activity is also substantially stimulated by certain 1,2-sn-diacylglycerols that bind specifically and stoichiometrically to a recognition site or sites on the enzyme. This site is called the diacylglycerol binding site, and it is located on the amino-terminal portion of protein kinase C, the so-called “regulatory domain”. The carboxy-terminal portion of protein kinase C carries the site at which protein phosphorylation is effected, and this portion is thus called the “kinase domain”.

Thus, the rate at which various protein kinase C family members carry out their enzymatic phosphorylation of certain substrates can be markedly enhanced by the presence of the cofactors such as phospholipids, diacylglycerols and, for some protein kinase C family members, calcium ions. This stimulation of protein kinase C activity is referred to as protein kinase C “activation”, and the activation of protein kinase C by the binding of diacylglycerols to the regulatory domain of protein kinase C is of particular importance in the normal and pathological functions of protein kinase C.

In contrast to the activation of protein kinase C, some chemical compounds have been shown, when added to protein kinase C enzyme assays, to reduce the rate at which protein kinase C phosphorylates its substrates; such compounds are referred to as protein kinase C “inhibitors” or, in some cases, “antagonists”. In some circumstances, protein kinase C inhibitors are capable of inhibiting various cellular or tissue phenomena which are thought to be mediated by protein kinase C.

Activation of protein kinase C by diacylglycerols has been shown to be an important physiological event that mediates the actions of a wide variety of hormones, neurotransmitters, and other biological control factors such as histamine, vasopressin, .alpha.-adrenergic agonists, dopamine agonists, muscarinic cholinergic agonists, platelet activating factor, etc. {see Y. Nishizuka, Nature 308: 693-698 (1984) and Science 225: 1365-1370 (1984) for reviews}.

The biological role of protein kinase C is also of great interest because of the discovery that certain very powerful tumor promoting chemicals activate this enzyme by binding specifically and with very high affinity to the diacylglycerol binding site on the enzyme. In addition to diacylglycerols, there are at present six other known classes of compounds that bind to this site: diterpenes such as the phorbol esters; indole alkaloids (indolactams) such as the teleocidins, lyngbyatoxin, and indolactam V; polyacetates such as the aplysiatoxins and oscillatoxins; certain derivatives of diaminobenzyl alcohol; macrocyclic lactones of the bryostatin class; and benzolactams such as (−)-BL-V8-310. The phorbol esters have long been known as powerful tumor promoters, the teleocidins and aplysiatoxins are now known to have this activity, and it appears likely that additional classes of compounds will be found to have the toxic and tumor promoting activities associated with the capability to bind to the diacylglycerol site of protein kinase C and thus activate the enzyme. Other toxicities of these agents when administered to animals include lung injury and profound changes in blood elements, such as leukopenia and neutropenia.

Compounds of the Invention

In certain embodiments, a compound of the present invention is represented by A:

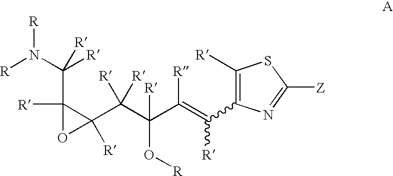

wherein

Z represents H, alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylakylamino, sulfhydryl, alkylthio, arylthio, arylakylthio, nitro, azido, alkylseleno, formyl, acyl, carboxyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₈₀;

R represents independently for each occurrence H, alkyl, cycloalkyl, alkenyl, aryl, heteroaryl, arylalkyl, trialkylsilyl, alkyldiarylsilyl, dialkylarylsilyl, triarylsilyl, formyl, acyl, alkylsulfonyl, arylsulfonyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;

R′ represents independently for each occurrence H, alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylakylamino, sulfhydryl, alkylthio, arylthio, arylakylthio, nitro, azido, alkylseleno, formyl, acyl, carboxyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₈₀;

R″ represents alkyl, cycloalkyl, alkenyl, aryl, heteroaryl, arylalkyl, formyl, acyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;

R₈₀ represents independently for each occurrence an aryl, cycloalkyl, cycloalkenyl, heterocyclyl, or polycyclyl moiety;

m is independently for each occurrence an integer in the range 0 to 8 inclusive;

the geometric configuration at an alkenyl moiety in a compound represented by A is E, Z, or a mixture thereof; and

the stereochemical configuration at a stereocenter in a compound represented by A is R, S, or a mixture thereof.

In certain embodiments, the compounds of the present invention are represented by A and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀.

In certain embodiments, the compounds of the present invention are represented by A and the attendant definitions, wherein R′ represents independently for each occurrence H, alkyl, cycloalkyl, alkenyl, aryl, heteroaryl, or arylalkyl.

In certain embodiments, the compounds of the present invention are represented by A and the attendant definitions, wherein R″ represents independently for each occurrence H, alkyl, cycloalkyl, alkenyl, aryl, heteroaryl, or arylalkyl.

In certain embodiments, the compounds of the present invention are represented by A and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; and R′ represents independently for each occurrence H, alkyl, cycloalkyl, alkenyl, aryl, heteroaryl, or arylalkyl.

In certain embodiments, the compounds of the present invention are represented by A and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; R′ represents independently for each occurrence H, alkyl, cycloalkyl, alkenyl, aryl, heteroaryl, or arylalkyl; and R″ represents independently for each occurrence H, alkyl, cycloalkyl, alkenyl, aryl, heteroaryl, or arylalkyl.

In an assay based on a mammalian GPCR or protein kinase, certain compounds according to structure A have IC₅₀ values less than 1 μM, more preferably less than 100 nM, and most preferably less than 10 nM.

In an assay based on a mammalian GPCR or protein kinase, certain compounds according to structure A have EC₅₀ values less than 1 μM, more preferably less than 100 nM, and most preferably less than 10 nM.

In certain embodiments, compounds according to structure A are effective in the treatment of mammals suffering from addiction, anxiety, depression, sexual dysfunction, hypertension, migraine, Alzheimer's disease, obesity, emesis, psychosis, analgesia, schizophrenia, Parkinson's disease, restless leg syndrome, sleeping disorders, attention deficit hyperactivity disorder, irritable bowel syndrome, premature ejaculation, menstrual dysphoria syndrome, urinary incontinence, inflammatory pain, neuropathic pain, Lesche-Nyhane disease, Wilson's disease, Tourette's syndrome, psychiatric disorders, stroke, senile dementia, peptic ulcers, pulmonary obstruction disorders, asthma, cancer, cell proliferative disorders, fibrotic disorders, metabolic disorders, or diabetes.

In certain embodiments, a compound of the present invention is represented by B:

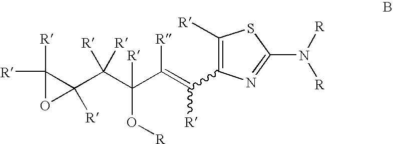

wherein

R represents independently for each occurrence H, alkyl, cycloalkyl, alkenyl, aryl, heteroaryl, arylalkyl, trialkylsilyl, alkyldiarylsilyl, dialkylarylsilyl, triarylsilyl, formyl, acyl, alkylsulfonyl, arylsulfonyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;

R′ represents independently for each occurrence H, alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylakylamino, sulfhydryl, alkylthio, arylthio, arylakylthio, nitro, azido, alkylseleno, formyl, acyl, carboxyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₈₀;

R″ represents alkyl, cycloalkyl, alkenyl, aryl, heteroaryl, arylalkyl, formyl, acyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;

R₈₀ represents independently for each occurrence an aryl, cycloalkyl, cycloalkenyl, heterocyclyl, or polycyclyl moiety;

m is independently for each occurrence an integer in the range 0 to 8 inclusive;

the geometric configuration at an alkenyl moiety in a compound represented by B is E, Z, or a mixture thereof; and

the stereochemical configuration at a stereocenter in a compound represented by B is R, S, or a mixture thereof.

In certain embodiments, the compounds of the present invention are represented by B and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀.

In certain embodiments, the compounds of the present invention are represented by B and the attendant definitions, wherein R′ represents independently for each occurrence H, alkyl, cycloalkyl, alkenyl, aryl, heteroaryl, or arylalkyl.

In certain embodiments, the compounds of the present invention are represented by B and the attendant definitions, wherein R″ represents independently for each occurrence H, alkyl, cycloalkyl, alkenyl, aryl, heteroaryl, or arylalkyl.

In certain embodiments, the compounds of the present invention are represented by B and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; and R′ represents independently for each occurrence H, alkyl, cycloalkyl, alkenyl, aryl, heteroaryl, or arylalkyl.

In certain embodiments, the compounds of the present invention are represented by B and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; R′ represents independently for each occurrence H, alkyl, cycloalkyl, alkenyl, aryl, heteroaryl, or arylalkyl; and R″ represents independently for each occurrence H, alkyl, cycloalkyl, alkenyl, aryl, heteroaryl, or arylalkyl.

In an assay based on a mammalian GPCR or protein kinase, certain compounds according to structure B have IC₅₀ values less than 1 μM, more preferably less than 100 nM, and most preferably less than 10 nM.

In an assay based on a mammalian GPCR or protein kinase, certain compounds according to structure B have EC₅₀ values less than 1 μM, more preferably less than 100 nM, and most preferably less than 10 nM.

In certain embodiments, compounds according to structure B are effective in the treatment of mammals suffering from addiction, anxiety, depression, sexual dysfunction, hypertension, migraine, Alzheimer's disease, obesity, emesis, psychosis, analgesia, schizophrenia, Parkinson's disease, restless leg syndrome, sleeping disorders, attention deficit hyperactivity disorder, irritable bowel syndrome, premature ejaculation, menstrual dysphoria syndrome, urinary incontinence, inflammatory pain, neuropathic pain, Lesche-Nyhane disease, Wilson's disease, Tourette's syndrome, psychiatric disorders, stroke, senile dementia, peptic ulcers, pulmonary obstruction disorders, asthma, cancer, cell proliferative disorders, fibrotic disorders, metabolic disorders, or diabetes.

In certain embodiments, a compound of the present invention is represented by C:

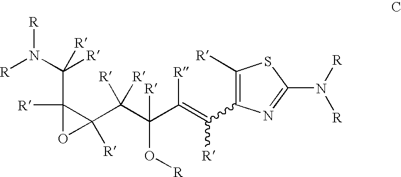

wherein

R represents independently for each occurrence H, alkyl, cycloalkyl, alkenyl, aryl, heteroaryl, arylalkyl, trialkylsilyl, alkyldiarylsilyl, dialkylarylsilyl, triarylsilyl, formyl, acyl, alkylsulfonyl, arylsulfonyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;

R′ represents independently for each occurrence H, alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylakylamino, sulfhydryl, alkylthio, arylthio, arylakylthio, nitro, azido, alkylseleno, formyl, acyl, carboxyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₈₀;

R″ represents alkyl, cycloalkyl, alkenyl, aryl, heteroaryl, arylalkyl, formyl, acyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;

R₈₀ represents independently for each occurrence an aryl, cycloalkyl, cycloalkenyl, heterocyclyl, or polycyclyl moiety;

m is independently for each occurrence an integer in the range 0 to 8 inclusive;

the geometric configuration at an alkenyl moiety in a compound represented by C is E, Z, or a mixture thereof; and

the stereochemical configuration at a stereocenter in a compound represented by C is R, S, or a mixture thereof.

In certain embodiments, the compounds of the present invention are represented by C and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀.

In certain embodiments, the compounds of the present invention are represented by C and the attendant definitions, wherein R′ represents independently for each occurrence H, alkyl, cycloalkyl, alkenyl, aryl, heteroaryl, or arylalkyl.

In certain embodiments, the compounds of the present invention are represented by C and the attendant definitions, wherein R″ represents independently for each occurrence H, alkyl, cycloalkyl, alkenyl, aryl, heteroaryl, or arylalkyl.

In certain embodiments, the compounds of the present invention are represented by C and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; and R′ represents independently for each occurrence H, alkyl, cycloalkyl, alkenyl, aryl, heteroaryl, or arylalkyl.

In certain embodiments, the compounds of the present invention are represented by C and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; R′ represents independently for each occurrence H, alkyl, cycloalkyl, alkenyl, aryl, heteroaryl, or arylalkyl; and R″ represents independently for each occurrence H, alkyl, cycloalkyl, alkenyl, aryl, heteroaryl, or arylalkyl.

In an assay based on a mammalian GPCR or protein kinase, certain compounds according to structure C have IC₅₀ values less than 1 μM, more preferably less than 100 nM, and most preferably less than 10 nM.

In an assay based on a mammalian GPCR or protein kinase, certain compounds according to structure C have EC₅₀ values less than 1 μM, more preferably less than 100 nM, and most preferably less than 10 nM.

In certain embodiments, compounds according to structure C are effective in the treatment of mammals suffering from addiction, anxiety, depression, sexual dysfunction, hypertension, migraine, Alzheimer's disease, obesity, emesis, psychosis, analgesia, schizophrenia, Parkinson's disease, restless leg syndrome, sleeping disorders, attention deficit hyperactivity disorder, irritable bowel syndrome, premature ejaculation, menstrual dysphoria syndrome, urinary incontinence, inflammatory pain, neuropathic pain, Lesche-Nyhane disease, Wilson's disease, Tourette's syndrome, psychiatric disorders, stroke, senile dementia, peptic ulcers, pulmonary obstruction disorders, asthma, cancer, cell proliferative disorders, fibrotic disorders, metabolic disorders, or diabetes.

In certain embodiments, a compound of the present invention is represented by D:

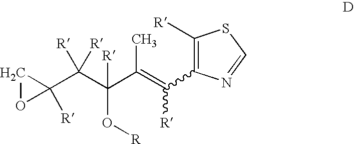

wherein

R represents H, alkyl, cycloalkyl, alkenyl, aryl, heteroaryl, arylalkyl, trialkylsilyl, alkyldiarylsilyl, dialkylarylsilyl, triarylsilyl, formyl, acyl, alkylsulfonyl, arylsulfonyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;

R′ represents independently for each occurrence H, alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylakylamino, sulfhydryl, alkylthio, arylthio, arylakylthio, nitro, azido, alkylseleno, formyl, acyl, carboxyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₈₀;

R₈₀ represents independently for each occurrence an aryl, cycloalkyl, cycloalkenyl, heterocyclyl, or polycyclyl moiety;

m is independently for each occurrence an integer in the range 0 to 8 inclusive;

the geometric configuration at an alkenyl moiety in a compound represented by D is E, Z, or a mixture thereof; and

the stereochemical configuration at a stereocenter in a compound represented by D is R, S, or a mixture thereof.

In certain embodiments, the compounds of the present invention are represented by D and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ-R₈₀.

In certain embodiments, the compounds of the present invention are represented by D and the attendant definitions, wherein R′ represents independently for each occurrence H, alkyl, cycloalkyl, alkenyl, aryl, heteroaryl, or arylalkyl.

In certain embodiments, the compounds of the present invention are represented by D and the attendant definitions, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; and R′ represents independently for each occurrence H, alkyl, cycloalkyl, alkenyl, aryl, heteroaryl, or arylalkyl.

In an assay based on a mammalian GPCR or protein kinase, certain compounds according to structure D have IC₅₀ values less than 1 μM, more preferably less than 100 nM, and most preferably less than 10 nM.

In an assay based on a mammalian GPCR or protein kinase, certain compounds according to structure D have EC₅₀ values less than 1 μM, more preferably less than 100 nM, and most preferably less than 10 nM.

In certain embodiments, compounds according to structure D are effective in the treatment of mammals suffering from addiction, anxiety, depression, sexual dysfunction, hypertension, migraine, Alzheimer's disease, obesity, emesis, psychosis, analgesia, schizophrenia, Parkinson's disease, restless leg syndrome, sleeping disorders, attention deficit hyperactivity disorder, irritable bowel syndrome, premature ejaculation, menstrual dysphoria syndrome, urinary incontinence, inflammatory pain, neuropathic pain, Lesche-Nyhane disease, Wilson's disease, Tourette's syndrome, psychiatric disorders, stroke, senile dementia, peptic ulcers, pulmonary obstruction disorders, asthma, cancer, cell proliferative disorders, fibrotic disorders, metabolic disorders, or diabetes.

In certain embodiments, the present invention relates to a compound represented by any of the structures outlined above, wherein said compound is a single stereoisomer.

In certain embodiments, the present invention relates to a formulation, comprising a compound represented by any of the structures outlined above; and a pharmaceutically acceptable excipient.

In certain embodiments, the present invention relates to ligands for a GPCR or a protein kinase, wherein the ligands are represented by any of the structures outlined above, and any of the sets of definitions associated with one of those structures. In certain embodiments, the ligands of the present invention are antagonists, agonists, partial agonists or inverse agonists of a GPCR or protein kinase. In any event, the ligands of the present invention preferably exert their effect on a GPCR or protein kinase at a concentration less than about 1 micromolar, more preferably at a concentration less than about 100 nanomolar, and most preferably at a concentration less than 10 nanomolar.

The compounds of the invention are indicated for use in the treatment of inflammatory, immunological, bronchopulmonary, cardiovascular, oncological or CNS-degenerative disorders; preferably for oral or topical treatment of inflammatory and/or immunological disorders, such as the oral or topical treatment of airway diseases involving inflammatory conditions, e.g. asthma, bronchitis; or atopic diseases, e.g. rhinitis or atopic dermatitis; inflammatory bowel diseases, e.g. Crohn's disease or colitis; autoimmune diseases e.g. multiple sclerosis, diabetes, atherosclerosis, psoriasis, systemic lupus erythematosus or rheumatoid arthritis; malignant diseases, e.g. skin or lung cancer; HIV infections or AIDS; or for inhibiting rejection of organs/transplants. The compounds of the invention are also indicated for use in treatment of heart failure, and in treatment of diabetic patients with macular edema or diabetic retinopathy.

A preferred embodiment of the invention is the treatment of a patient having inflammatory pain. For example, administration of certain kinase inhibitors significantly diminishes both acute and chronic hyperalgesia resulting from exposure to the inflammatory agent carrageenan; moreover, administration of certain kinase inhibitors diminishes hyperalgesia due to diabetes, chemotherapy or traumatic nerve injury. Such inflammatory pain may be acute or chronic and can be due to any number of conditions characterized by inflammation including, without limitation, sunburn, rheumatoid arthritis, osteoarthritis, colitis, carditis, dermatitis, myositis, neuritis and collagen vascular diseases. In addition, administration of a compound of the present invention to a subject immediately prior to, during or after an inflammatory event can ameliorate both the acute pain and the chronic hyperalgesia that the subject would otherwise experience.

Another preferred embodiment of the invention is the treatment of a patient having neuropathic pain. Such patients can have a neuropathy classified as a radiculopathy, mononeuropathy, mononeuropathy multiplex, polyneuropathy or plexopathy. Diseases in these classes can be caused by a variety of nerve-damaging conditions or procedures, including, without limitation, trauma, stroke, demyelinating diseases, abscess, surgery, amputation, inflammatory diseases of the nerves, causalgia, diabetes, collagen vascular diseases, trigeminal neuralgia, rheumatoid arthritis, toxins, cancer (which can cause direct or remote (e.g. paraneoplastic) nerve damage), chronic alcoholism, herpes infection, AIDS, and chemotherapy. Nerve damage causing hyperalgesia can be in peripheral or CNS nerves. This embodiment of the invention is based on the fact that administration of certain kinase inhibitors significantly diminishes hyperalgesia due to diabetes, chemotherapy or traumatic nerve injury.

Another aspect of the invention is a method of identifying a compound that modulates pain, by selecting, as a test compound, a compound that modulates the activity of a protein kinase, and administering said test compound to a subject to determine whether pain is modulated. Preferably, the compound will inhibit the activity of a protein kinase, and the subject will be an animal commonly used in pain research and/or development. The ability of a test compound to inhibit, enhance or modulate the activity of a protein kinase may be determined with suitable assays measuring the activity of the protein kinase. For example, responses such as its activity, e.g., enzymatic activity, or the kinase's ability to bind its ligand, adapter molecule or substrate may be determined in in vitro assays. Cellular assays can be developed to monitor a modulation of second messenger production, changes in cellular metabolism, or effects on enzymatic activity. These assays may be performed using conventional techniques developed for these purposes. Finally, the ability of a test compound to inhibit, enhance or modulate the function of a protein kinase will be measured in suitable animal models in vivo.

Preferred embodiments of the present invention include a composition combining an inhibitor of a protein kinase with one or more additional pain-reducing agents and a method of administering such a composition. An individual pain medication often provides only partially effective pain alleviation because it interferes with just one pain-transducing pathway out of many. Alternatively, protein kinase inhibitors can be administered in combination with a pain-reducing (analgesic) agent that acts at a different point in the pain perception process. A protein kinase inhibitor can minimize pain by altering the responses of nociceptors to noxious stimuli. One class of analgesics, such as NSAIDs, down-regulates the chemical messengers of the stimuli that are detected by the nociceptors and another class of drugs, such as opioids, alters the processing of nociceptive information in the CNS. Other analgesics are local anesthetics, anticonvulsants, and antidepressants. Administering one or more classes of drug in addition to PKC inhibitors can provide more effective amelioration of pain. NSAIDs are preferred components of the composition of the invention. Preferred NSAIDs are aspirin, acetaminophen, ibuprofen, and indomethacin.

Another aspect of the invention is directed to compounds which modulate protein kinase signal transduction by affecting the enzymatic activity of a protein kinase, and thereby interfering with the signal transduced by such proteins. More particularly, the present invention is directed to compounds which modulate the protein kinase mediated signal transduction pathways as a therapeutic approach to cure many kinds of solid tumors, including but not limited to carcinoma, sarcoma, erythroblastoma, glioblastoma, meningioma, astrocytoma, melanoma and myoblastoma. Indications may include, but are not limited to brain cancers, bladder cancers, ovarian cancers, gastric cancers, pancreas cancers, colon cancers, blood cancers, lung cancers, bone cancers and leukemias.

Further examples, without limitation, of the types of disorders related to unregulated protein kinase activity that the compounds described herein may be useful in preventing, treating and studying, are cell proliferative disorders, fibrotic disorders and metabolic disorders. Cell proliferative disorders which may be prevented, treated or further studied by the present invention include cancers, blood vessel proliferative disorders and mesangial cell proliferative disorders.

Blood vessel proliferative disorders refer to angiogenic and vasculogenic disorders generally resulting in abnormal proliferation of blood vessels. The formation and spreading of blood vessels, or vasculogenesis and angiogenesis, respectively, play important roles in a variety of physiological processes such as embryonic development, corpus luteum formation, wound healing and organ regeneration. They also play a pivotal role in cancer development. Other examples of blood vessel proliferation disorders include arthritis, where new capillary blood vessels invade the joint and destroy cartilage, and ocular diseases, like diabetic retinopathy, where new capillaries in the retina invade the vitreous, bleed and cause blindness. Conversely, disorders related to the shrinkage, contraction or closing of blood vessels, such as restenosis, are also implicated.

Fibrotic disorders refer to the abnormal formation of extracellular matrices. Examples of fibrotic disorders include hepatic cirrhosis and mesangial cell proliferative disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis. Other fibrotic disorders implicated include atherosclerosis.

Mesangial cell proliferative disorders refer to disorders brought about by abnormal proliferation of mesangial cells. Mesangial proliferative disorders include various human renal diseases, such as glomerulonephritis, diabetic nephropathy, malignant nephrosclerosis, thrombotic microangiopathy syndromes, transplant rejection, and glomerulopathies. For instance, PDGFR has been implicated in the maintenance of mesangial cell proliferation. Floege et al., 1993, Kidney International 43:47S-54S.

As noted previously, protein kinases have been associated with cell proliferative disorders. For example, some members of the family have been associated with the development of cancer. Some of these receptors, like EGFR (Tuzi et al., 1991, Br. J. Cancer 63:227-233; Torp et al., 1992, APMIS 100:713-719); HER2/neu (Slamon et al., 1989, Science 244:707-712) and PDGFR (Kumabe et al., 1992, Oncogene, 7:627-633) are over-expressed in many tumors and/or are persistently activated by autocrine loops. In fact, in the most common and severe cancers these receptor over-expressions and autocrine loops have been demonstrated (Akbasak and Suner-Akbasak et al., 1992, J. Neurol. Sci., 111:119-133; Dickson et al., 1992, Cancer Treatment Res. 61:249-273; Korc et al., 1992, J. Clin. Invest. 90:1352-1360); (Lee and Donoghue, 1992, J. Cell. Biol., 118:1057-1070; Korc et al., supra; Akbasak and Suner-Akbasak et al., supra). For example, the EGFR receptor has been associated with squamous cell carcinoma, astrocytoma, glioblastoma, head and neck cancer, lung cancer and bladder cancer. HER2 has been associated with breast, ovarian, gastric, lung, pancreas and bladder cancer. PDGFR has been associated with glioblastoma, lung, ovarian, melanoma and prostate. The protein kinase c-met has been generally associated with hepatocarcinogenesis and thus hepatocellular carcinoma. Additionally, c-met has been linked to malignant tumor formation. More specifically, c-met has been associated with, among other cancers, colorectal, thyroid, pancreatic and gastric carcinoma, leukemia and lymphoma. Additionally, over-expression of the c-met gene has been detected in patients with Hodgkins disease, Burkitts disease, and the lymphoma cell line.

IGF-IR, in addition to being implicated in nutritional support and in type-II diabetes, has also been associated with several types of cancers. For example, IGF-I has been implicated as an autocrine growth stimulator for several tumor types, e.g. human breast cancer carcinoma cells (Arteaga et al., 1989, J. Clin. Invest. 84:1418-1423) and small lung tumor cells (Macauley et al., 1990, Cancer Res., 50:2511-2517). In addition, IGF-I, while being integrally involved in the normal growth and differentiation of the nervous system, appears to be an autocrine stimulator of human gliomas. Sandberg-Nordqvist et al., 1993, Cancer Res. 53:2475-2478. The importance of the IGF-IR and its ligands in cell proliferation is further supported by the fact that many cell types in culture (fibroblasts, epithelial cells, smooth muscle cells, T-lymphocytes, myeloid cells, chondrocytes, osteoblasts, the stem cells of the bone marrow) are stimulated to grow by IGF-I. Goldring and Goldring, 1991, Eukarvotic Gene Expression,1:301-326. In a series of recent publications, Baserga even suggests that IGF-IR plays a central role in the mechanisms of transformation and, as such, could be a preferred target for therapeutic interventions for a broad spectrum of human malignancies. Baserga, 1995, Cancer Res., 55:249-252; Baserga, 1994, Cell 79:927-930; Coppola et al., 1994, Mol. Cell. Biol., 14:4588-4595.

The association between abnormal protein kinase activity and disease are not restricted to cancer, however. For example, protein kinases have been associated with metabolic diseases like psoriasis, diabetes mellitus, wound healing, inflammation, and neurodegenerative diseases. For example, EGFR has been indicated in corneal and dermal wound healing. Defects in the Insulin-R and IGF-1R are indicated in type-II diabetes mellitus. A more complete correlation between specific protein kinases and their therapeutic indications is set forth in Plowman et al., 1994, DN&P 7:334-339.

As noted previously, protein kinases including, but not limited to, src, abl, fps, yes, fyn, lyn, lck, blk, hck, fgr and yrk (reviewed by Bolen et al., 1992, FASEB J., 6:3403-3409) are involved in the proliferative and metabolic signal transduction pathway and thus were expected, and have been shown, to be involved in many protein-kinase-mediated disorders to which the present invention is directed. For example, mutated src (v-src) has been demonstrated as an oncoprotein (pp60.sup.c-src) in chicken. Moreover, its cellular homolog, the proto-oncogene pp60.sup.c-src transmits oncogenic signals of many receptors. For example, over-expression of EGFR or HER2/neu in tumors leads to the constitutive activation of p p60.sup.c-src, which is characteristic for the malignant cell but absent from the normal cell. On the other hand, mice deficient in the expression of c-src exhibit an osteopetrotic phenotype, indicating a key participation of c-src in osteoclast function and a possible involvement in related disorders. Similarly, Zap70 is implicated in T-cell signaling. Finally, protein kinases are likely involved in hyperimmune disorders.

In sum, the compounds of the present invention and salts, especially pharmaceutically acceptable salts, and solvates thereof, and solvates of such salts, are useful because they demonstrate pharmacological activity. In particular, they demonstrate activity as kinase inhibitors. Granet, R. A. et al, Analyt. Biochem. 1987; 163, 458-463; Olsson, H. et al, Cell Signal 1989, 1, 405-410; and Chakravarthy, B. R. et al, Analyt. Biochem. 1991, 196, 144-150.

Biochemical Activity at Cellular Receptors, and Assays to Detect That Activity

Assaying processes are well known in the art in which a reagent is added to a sample, and measurements of the sample and reagent are made to identify sample attributes stimulated by the reagent. For example, one such assay process concerns determining in a chromogenic assay the amount of an enzyme present in a biological sample or solution. Such assays are based on the development of a colored product in the reaction solution. The reaction develops as the enzyme catalyzes the conversion of a colorless chromogenic substrate to a colored product.

Another assay useful in the present invention concerns determining the ability of a ligand to bind to a biological receptor utilizing a technique well known in the art referred to as a radioligand binding assay. This assay accurately determines the specific binding of a radioligand to a targeted receptor through the delineation of its total and nonspecific binding components. Total binding is defined as the amount of radioligand that remains following the rapid separation of the radioligand bound in a receptor preparation (cell homogenates or recombinate receptors) from that which is unbound. The nonspecific binding component is defined as the amount of radioligand that remains following separation of the reaction mixture consisting of receptor, radioligand and an excess of unlabeled ligand. Under this condition, the only radioligand that remains represents that which is bound to components other that receptor. The specific radioligand bound is determined by subtracting the nonspecific from total radioactivity bound. For a specific example of radioligand binding assay for μ-opioid receptor, see Wang, J. B. et al. FEBS Letters 1994, 338, 217.

Assays useful in the present invention concern determining the activity of receptors the activation of which initiates subsequent intracellular events in which intracellular stores of calcium ions are released for use as a second messenger. Activation of some G-protein-coupled receptors stimulates the formation of inositol triphosphate (IP3, a G-protein-coupled receptor second messenger) through phospholipase C-mediated hydrolysis of phosphatidylinositol, Berridge and Irvine (1984). Nature 312:315-21. IP3 in turn stimulates the release of intracellular calcium ion stores.

A change in cytoplasmic calcium ion levels caused by release of calcium ions from intracellular stores is used to determine G-protein-coupled receptor function. This is another type of indirect assay. Among G-protein-coupled receptors are muscarinic acetylcholine receptors (mAChR), adrenergic receptors, sigma receptors, serotonin receptors, dopamine receptors, angiotensin receptors, adenosine receptors, bradykinin receptors, metabotropic excitatory amino acid receptors and the like. Cells expressing such G-protein-coupled receptors may exhibit increased cytoplasmic calcium levels as a result of contribution from both intracellular stores and via activation of ion channels, in which case it may be desirable although not necessary to conduct such assays in calcium-free buffer, optionally supplemented with a chelating agent such as EGTA, to distinguish fluorescence response resulting from calcium release from internal stores. Another type of indirect assay involves determining the activity of receptors which, when activated, result in a change in the level of intracellular cyclic nucleotides, e.g., cAMP, cGMP. For example, activation of some dopamine, serotonin, metabotropic glutamate receptors and muscarinic acetylcholine receptors results in a decrease in the cAMP or cGMP levels of the cytoplasm.

Furthermore, there are cyclic nucleotide-gated ion channels, e.g., rod photoreceptor cell channels and olfactory neuron channels [see, Altenhofen, W. et al. (1991) Proc. Natl. Acad. Sci U.S.A. 88:9868-9872 and Dhallan et al. (1990) Nature 347:184-187] that are permeable to cations upon activation by binding of cAMP or cGMP. A change in cytoplasmic ion levels caused by a change in the amount of cyclic nucleotide activation of photo-receptor or olfactory neuron channels is used to determine function of receptors that cause a change in cAMP or cGMP levels when activated. In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. Cell for this type of assay can be made by co-transfection of a host cell with DNA encoding a cyclic nucleotide-gated ion channel and a DNA encoding a receptor (e.g., certain metabotropic glutamate receptors, muscarinic acetylcholine receptors, dopamine receptors, serotonin receptors and the like, which, when activated, causes a change in cyclic nucleotide levels in the cytoplasm.

Any cell expressing a receptor protein which is capable, upon activation, of directly increasing the intracellular concentration of calcium, such as by opening gated calcium channels, or indirectly affecting the concentration of intracellular calcium as by causing initiation of a reaction which utilizes Ca<2+> as a second messenger (e.g., G-protein-coupled receptors), may form the basis of an assay. Cells endogenously expressing such receptors or ion channels and cells which may be transfected with a suitable vector encoding one or more such cell surface proteins are known to those of skill in the art or may be identified by those of skill in the art. Although essentially any cell which expresses endogenous ion channel and/or receptor activity may be used, it is preferred to use cells transformed or transfected with heterologous DNAs encoding such ion channels and/or receptors so as to express predominantly a single type of ion channel or receptor. Many cells that may be genetically engineered to express a heterologous cell surface protein are known. Such cells include, but are not limited to, baby hamster kidney (BHK) cells (ATCC No. CCL10), mouse L cells (ATCC No. CCLL3), DG44 cells [see, Chasin (1986) Cell. Molec. Genet. 12:555] human embryonic kidney (HEK) cells (ATCC No. CRL1573), Chinese hamster ovary (CHO) cells (ATCC Nos. CRL9618, CCL61, CRL9096), PC12 cells (ATCC No. CRL1721) and COS-7 cells (ATCC No. CRL1651). Preferred cells for heterologous cell surface protein expression are those that can be readily and efficiently transfected. Preferred cells include HEK 293 cells, such as those described in U.S. Pat. No. 5,024,939.

Any compound which is known to activate ion channels or receptors of interest may be used to initiate an assay. Choosing an appropriate ion channel- or receptor-activating reagent depending on the ion channel or receptor of interest is within the skill of the art. Direct depolarization of the cell membrane to determine calcium channel activity may be accomplished by adding a potassium salt solution having a concentration of potassium ions such that the final concentration of potassium ions in the cell-containing well is in the range of about 50-150 mM (e.g., 50 mM KCl). With respect to ligand-gated receptors and ligand-gated ion channels, ligands are known which have affinity for and activate such receptors. For example, nicotinic acetyloholine receptors are known to be activated by nicotine or acetylcholine; similarly, muscarinic and acetylcholine receptors may be activated by addition of muscarine or carbamylcholine.

Agonist assays may be carried out on cells known to possess ion channels and/or receptors to determine what effect, if any, a compound has on activation or potentiation of ion channels or receptors of interest. Agonist assays also may be carried out using a reagent known to possess ion channel- or receptor-activating capacity to determine whether a cell expresses the respective functional ion channel or receptor of interest.

Contacting a functional receptor or ion channel with agonist typically activates a transient reaction; and prolonged exposure to an agonist may desensitize the receptor or ion channel to subsequent activation. Thus, in general, assays for determining ion channel or receptor function should be initiated by addition of agonist (i.e., in a reagent solution used to initiate the reaction). The potency of a compound having agonist activity is determined by the detected change in some observable in the cells (typically an increase, although activation of certain receptors causes a decrease) as compared to the level of the observable in either the same cell, or substantially identical cell, which is treated substantially identically except that reagent lacking the agonist (i.e., control) is added to the well. Where an agonist assay is performed to test whether or not a cell expresses the functional receptor or ion channel of interest, known agonist is added to test-cell-containing wells and to wells containing control cells (substantially identical cell that lacks the specific receptors or ion channels) and the levels of observable are compared. Depending on the assay, cells lacking the ion channel and/or receptor of interest should exhibit substantially no increase in observable in response to the known agonist. A substantially identical cell may be derived from the same cells from which recombinant cells are prepared but which have not been modified by introduction of heterologous DNA. Alternatively, it may be a cell in which the specific receptors or ion channels are removed. Any statistically or otherwise significant difference in the level of observable indicates that the test compound has in some manner altered the activity of the specific receptor or ion channel or that the test cell possesses the specific functional receptor or ion channel.

In an example of drug screening assays for identifying compounds which have the ability to modulate ion channels or receptors of interest, individual wells (or duplicate wells, etc.) contain a distinct cell type, or distinct recombinant cell line expressing a homogeneous population of a receptor or ion channel of interest, so that the compound having unidentified activity may be screened to determine whether it possesses modulatory activity with respect to one or more of a variety of functional ion channels or receptors. It is also contemplated that each of the individual wells may contain the same cell type so that multiple compounds (obtained from different reagent sources in the apparatus or contained within different wells) can be screened and compared for modulating activity with respect to one particular receptor or ion channel type.

Antagonist assays, including drug screening assays, may be carried out by incubating cells having functional ion channels and/or receptors in the presence and absence of one or more compounds, added to the solution bathing the cells in the respective wells of the microtiter plate for an amount of time sufficient (to the extent that the compound has affinity for the ion channel and/or receptor of interest) for the compound(s) to bind to the receptors and/or ion channels, then activating the ion channels or receptors by addition of known agonist, and measuring the level of observable in the cells as compared to the level of observable in either the same cell, or substantially identical cell, in the absence of the putative antagonist.

The assays are thus useful for rapidly screening compounds to identify those that modulate any receptor or ion channel in a cell. In particular, assays can be used to test functional ligand-receptor or ligand-ion channel interactions for cell receptors including ligand-gated ion channels, voltage-gated ion channels, G-protein-coupled receptors and growth factor receptors.

Those of ordinary skill in the art will recognize that assays may encompass measuring a detectable change of a solution as a consequence of a cellular event which allows a compound, capable of differential characteristics, to change its characteristics in response to the cellular event. By selecting a particular compound which is capable of differential characteristics upon the occurrence of a cellular event, various assays may be performed. For example, assays for determining the capacity of a compound to induce cell injury or cell death may be carried out by loading the cells with a pH-sensitive fluorescent indicator such as BCECF (Molecular Probes, Inc., Eugene, Oreg. 97402, Catalog #B1150) and measuring cell injury or cell death as a function of changing fluorescence over time.

In a further example of useful assays, the function of receptors whose activation results in a change in the cyclic nucleotide levels of the cytoplasm may be directly determined in assays of cells that express such receptors and that have been injected with a fluorescent compound that changes fluorescence upon binding cAMP. The fluorescent compound comprises cAMP-dependent-protein kinase in which the catalytic and regulatory subunits are each labelled with a different fluorescent-dye [Adams et al. (1991) Nature 349:694-697]. When cAMP binds to the regulatory subunits, the fluorescence emission spectrum changes; this change can be used as an indication of a change in cAMP concentration.

The function of certain neurotransmitter transporters which are present at the synaptic cleft at the junction between two neurons may be determined by the development of fluorescence in the cytoplasm of such neurons when conjugates of an amine acid and fluorescent indicator (wherein the fluorescent indicator of the conjugate is an acetoxymethyl ester derivative e.g., 5-(aminoacetamido)fluorescein; Molecular Probes, Catalog #A1363) are transported by the neurotransmitter transporter into the cytoplasm of the cell where the ester group is cleaved by esterase activity and the conjugate becomes fluorescent.

In practicing an assay of this type, a reporter gene construct is inserted into an eukaryotic cell to produce a recombinant cell which has present on its surface a cell surface protein of a specific type. The cell surface receptor may be endogenously expressed or it may be expressed from a heterologous gene that has been introduced into the cell. Methods for introducing heterologous DNA into eukaryotic cells are-well known in the art and any such method may be used. In addition, DNA encoding various cell surface proteins is known to those of skill in the art or it may be cloned by any method known to those of skill in the art.

The recombinant cell is contacted with a test compound and the level of reporter gene expression is measured. The contacting may be effected in any vehicle and the testing may be by any means using any protocols, such as serial dilution, for assessing specific molecular interactions known to those of skill in the art. After contacting the recombinant cell for a sufficient time to effect any interactions, the level of gene expression is measured. The amount of time to effect such interactions may be empirically determined, such as by running a time course and measuring the level of transcription as a function of time. The amount of transcription may be measured using any method known to those of skill in the art to be suitable. For example, specific mRNA expression may be detected using Northern blots or specific protein product may be identified by a characteristic stain. The amount of transcription is then compared to the amount of transcription in either the same cell in the absence of the test. compound or it may be compared with the amount of transcription in a substantially identical cell that lacks the specific receptors. A substantially identical cell may be derived from the same cells from which the recombinant cell was prepared but which had not been modified by introduction of heterologous DNA. Alternatively, it may be a cell in which the specific receptors are removed. Any statistically or otherwise significant difference in the amount of transcription indicates that the test compound has in some manner altered the activity of the specific receptor.

If the test compound does not appear to enhance, activate or induce the activity of the cell surface protein, the assay may be repeated and modified by the introduction of a step in which the recombinant cell is first tested for the ability of a known agonist or activator of the specific receptor to activate transcription if the transcription is induced, the test compound is then assayed for its ability to inhibit, block or otherwise affect the activity of the agonist.

The transcription based assay is useful for identifying compounds that interact with any cell surface protein whose activity ultimately alters gene expression. In particular, the assays can be used to test functional ligand-receptor or ligand-ion channel interactions for a number of categories of cell surface-localized receptors, including: ligand-gated ion channels and voltage-gated ion channels, and G protein-coupled receptors.

Any transfectable cell that can express the desired cell surface protein in a manner such the protein functions to intracellularly transduce an extracellular signal may be used. The cells may be selected such that they endogenously express the cell surface protein or may be genetically engineered to do so. Many such cells are known to those of skill in the art. Such cells include, but are not limited to Ltk<−> cells, PC12 cells and COS-7 cells.

The preparation of cells which express a receptor or ion channel and a reporter gene expression construct, and which are useful for testing compounds to assess their activities, is exemplified in the Examples provided herewith by reference to mammalian Ltk<−> and COS-7 cell lines, which express the Type I human muscarinic (HM1) receptor and which are transformed with either a c-fos promoter-CAT reporter gene expression construct or a c-fos promoter-luciferase reporter gene expression construct.

Any cell surface protein that is known to those of skill in the art or that may be identified by those of skill in the art may used in the assay. The cell surface protein may endogenously expressed on the selected cell or it may be expressed from cloned DNA. Exemplary cell surface proteins include, but are not limited to, cell surface receptors and ion channels. Cell surface receptors include, but are not limited to, muscarinic receptors (e.g., human M2 (GenBank accession #M16404); rat M3 (GenBank accession #M16407); human M4 (GenBank accession #M16405); human M5 (Bonner et al. (1988) Neuron 1:403-410); and the like); neuronal nicotinic acetylcholine receptors (e.g., the alpha 2, alpha 3 and beta 2 subtypes disclosed in U.S. Ser. No. 504,455 (filed Apr. 3, 1990), hereby expressly incorporated by reference herein in its entirety); the rat alpha 2 subunit (Wada et al. (1988) Science 240:330-334); the rat alpha 3 subunit (Boulter et al. (1986) Nature 319:368-374); the rat alpha 4 subunit (Goldman et al. (1987) cell 48:965-973); the rat alpha 5 subunit (Boulter et al. (1990) J. Biol. Chem. 265:4472-4482); the rat beta 2 subunit (Deneris et al. (1988) Neuron 1:45-54); the rat beta 3 subunit (Deneris et al. (1989) J. Biol. Chem. 264: 6268-6272); the rat beta 4 subunit (Duvoisin et al. (1989) Neuron 3:487-496); combinations of the rat alpha subunits, beta subunits and alpha and beta subunits; GABA receptors (e.g., the bovine alpha 1 and beta 1 subunits (Schofield et al. (1987) Nature 328:221-227); the bovine alpha 2 and alpha 3 subunits (Levitan et al. (1988) Nature 335:76-79); the gamma-subunit (Pritchett et al. (1989) Nature 338:582-585); the beta 2 and beta 3 subunits (Ymer et alo (1989) EMBO J. 8:1665-1670); the delta subunit (Shivers, B. D. (1989) Neuron 3:327-337); and the like); glutamate receptors (e.g., receptor isolated from rat brain (Hollmann et al. (1989) Nature 342:643-648); and the like); adrenergic receptors (e.g., human beta 1 (Frielle et al. (1987) Proc. Natl. Acad. Sci. 84.:7920-7924); human alpha 2 (Kobilka et al. (1987) Science 238:650-656); hamster beta 2 (Dixon et al. (1986) Nature 321:75-79); and the like); dopamine receptors (e.g., human D2 (Stormann et al. (1990) Molec. Pharm.37:1-6); rat (Bunzow et al. (1988) Nature 336:783-787); and the like); NGF receptors (e.g., human NGF receptors (Johnson et al. (1986) Cell 47:545-554); and the like); serotonin receptors (e.g., human 5HT1a (Kobilka et al. (1987) Nature 329:75-79); rat 5HT2 (Julius et al. (1990) PNAS 87:928-932); rat 5HT1c (Julius et al. (1988) Science 241:558-564); and the like).

Reporter gene constructs are prepared by operatively linking a reporter gene with at least one transcriptional regulatory element. If only one transcriptional regulatory element is included it must be a regulatable promoter, At least one of the selected transcriptional regulatory elements must be indirectly or directly regulated by the activity of the selected cell-surface receptor whereby activity of the receptor can be monitored via transcription of the reporter genes.

The construct may contain additional transcriptional regulatory elements, such as a FIRE sequence, or other sequence, that is not necessarily regulated by the cell surface protein, but is selected for its ability to reduce background level transcription or to amplify the transduced signal and to thereby increase the sensitivity and reliability of the assay.

Many reporter genes and transcriptional regulatory elements are known to those of skill in the art and others may be identified or synthesized by methods known to those of skill in the art.

A reporter gene includes any gene that expresses a detectable gene product, which may be RNA or protein. Preferred reporter genes are those that are readily detectable. The reporter gene may also be included in the construct in the form of a fusion gene with a gene that includes desired transcriptional regulatory sequences or exhibits other desirable properties.

Examples of reporter genes include, but are not limited to CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101).

Transcriptional control elements include, but are not limited to, promoters, enhancers, and repressor and activator binding sites, Suitable transcriptional regulatory elements may be derived from the transcriptional regulatory regions of genes whose expression is rapidly induced, generally within minutes, of contact between the cell surface protein and the effector protein that modulates the activity of the cell surface protein. Examples of such genes include, but are not limited to, the immediate early genes (see, Sheng et al. (1990) Neuron 4: 477-485), such as c-fos, Immediate early genes are genes that are rapidly induced upon binding of a ligand to a cell surface protein. The transcriptional control elements that are preferred for use in the gene constructs include transcriptional control elements from immediate early genes, elements derived from other genes that exhibit some or all of the characteristics of the immediate early genes, or synthetic elements that are constructed such that genes in operative linkage therewith exhibit such characteristics. The characteristics of preferred genes from which the transcriptional control elements are derived include, but are not limited to, low or undetectable expression in quiescent cells, rapid induction at the transcriptional level within minutes of extracellular simulation, induction that is transient and independent of new protein synthesis, subsequent shut-off of transcription requires new protein synthesis, and mRNAs transcribed from these genes have a short half-life. It is not necessary for all of these properties to be present.

Biochemical Activity at Protein Kinases, and Assays to Detect That Activity

Generally, a protein kinase of interest may be exposed to known agonists, known antagonists, and/or test compounds which may be, or may contain, agonists or antagonists. An agonist, antagonist, or test compound may be a chemical compound, a mixture of chemical compounds, a biological macromolecule, or an extract made from biological materials such as bacteria, plants, fungi, or animal cells or tissues. Test compounds are evaluated for potential activity as agonists or antagonists of the kinase by inclusion in screening assays described herein. An “agonist” enhances the activity of a receptor; an “antagonist” diminishes the activity of a receptor. The terms “agonist” and “antagonist”, as used herein, do not imply a particular mechanism of function.

The most widely used technique for measuring protein kinase activity is based on radioactive detection. In this method, a sample containing the kinase of interest is incubated with activators and a substrate in the presence of gamma ³²P-ATP. After a suitable incubation period, the reaction is stopped and an aliquot of the reaction mixture is placed directly onto a filter which binds the substrate. The filter is then washed multiple times to remove excess radioactivity, and the amount of radiolabelled phosphate incorporated into the substrate is measured by scintillation counting. This method is widely used and provides an accurate method for determining protein kinase activity in both crude and purified samples. However, because of the necessity of multiple washings, which are generally done by manually transferring the filter to a beaker and washing and rinsing with gentle agitation, the procedure is quite time consuming.

Other methods for detecting kinase activity are based on separations due to the charge differences between phosphorylated and non-phosphorylated proteins and peptides. In these respects, techniques based on gel electrophoresis and HPLC have, among others, been used. In combination with these techniques, spectrophotometric and fluorometric detection have been used. International Patent Application WO 93/10461 and U.S. Pat. Nos. 5,120,644 and 5,141,852 provide descriptions of many methods heretofore used for detecting protein kinase activity. Also reference is directed to Analytical Biochemistry, 209, 348-353, 1993, “Protein Kinase Assay Using Tritiated Peptide Substrates and Ferric Adsorbent Paper for Phosphopeptide Binding.”

More recent methods utilize a standard enzyme-linked immunosorbent assay (ELISA) for measuring kinase activity. These methods utilize purified heterologous substrate protein or synthetic substrate peptides anchored to a microtiter plate. After exposure of the substrate molecule to a sample containing the appropriate kinase, the level of phosphorylation is evaluated with antiphosphotyrosine antibodies to quantitate the amount of phosphorylated protein bound to the plate. The obvious limitation of this type of assay is that the activity of a kinase specific for the particular substrate used, is the only activity detected. Additionally, methods such as protein tyrosine kinase enzyme assays are unable to eliminate as potential drug candidates, inhibitors which are not cell permeable and, therefore, are not good choices for therapeutic agents.

Hirth et al., U.S. Pat. No. 5,763,198, for example, describes an ELISA-type assay in which a substrate-specific antibody is used as an anchoring molecule to isolate a protein substrate from a cell lysate preparation and immobilize it on a solid phase support. Hirth's method then determines the level of kinase activity by evaluating the tyrosine phosphorylation state of the protein substrate bound to the solid phase using an anti-phosphotyrosine antibody as the detecting molecule. Other methods for measuring tyrosine kinase activity, particularly tyrosine kinase receptor activity, are described in WO95/04136, EP 0 730 740 B1, and U.S. Pat. No. 5,599,681.

Compounds can be tested for PKC inhibitor or activator activity by adding them to growth medium containing mammalian PKC expressing-yeast, incubating the treated cells and determining the effect of the treatment on the growth rate of the cells. The growth rate of the treated cells can be compared to an appropriate control, e.g., untreated PKC-expressing yeast cells. More than one compound can be added to the yeast to investigate the joint action of more than one compound on the growth rate. The tests can be performed on small tubes or in multi-well plates and can be adopted for automated analysis by methods known to those skilled in the art. Since yeast growth can be easily monitored in a small test tube or on multi-well plates and a result is usually obtained within hours to days, this screen is suitable for large scale product screening. It can be applied to test the potential of any cosmetic or pharmaceutical product as protein kinase agonists or antagonists.

Prodrugs and Intermediates

It will be appreciated by those skilled in the art that, although certain protected derivatives of the compounds of the present invention, which may be made prior to a final deprotection stage, may not possess pharmacological activity as such, they may be administered parenterally or orally and thereafter metabolized in the body to form compounds of the invention which are pharmacologically active. Such derivatives may therefore be described as “prodrugs”. Moreover, certain compounds of the present invention may act as prodrugs of other compounds of the present invention. Critically, all prodrugs of compounds of the present invention are included within the scope of the present invention. Novel intermediates as described herein and their use in the manufacture of other compounds of the present invention also form part of the invention.

Methods to Prepare the Compounds of the Invention

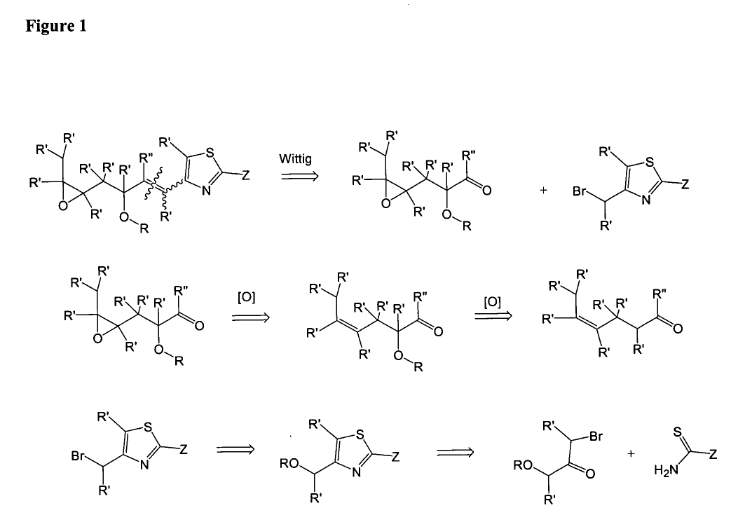

The compounds of the invention may be prepared by simple modification of known procedures. A retrosynthetic analysis is shown in FIG. 1. This strategy involves late-stage attachment of the thiazole moiety to the epoxide fragment. This strategy is considered to be advantageous because the thiazole fragment or the epoxide fragment can easily be modified to install additional functional groups before the two fragments are joined. It is contemplated that the thiazole moiety could be coupled with the ketone (or aldehyde) fragment via a Wittig reaction. The terminal expoxide could be formed from expoxidation of a terminal alkene using dioxirane or a percarboxylic acid. The α-hydroxy ketone could be prepared by reaction of an enone with MoO₅ or Davis oxaziridine. See, e.g., Anderson et al. Synlett 1990, 107; and Davis et al. J. Org. Chem. 1984, 49, 3241. Alternatively, the α-hydroxy ketone could be prepared by reaction of its silylenolether derivative with m-chloroperbenzoic acid. See, e.g., J. March, Advanced Organic Chemistry, McGraw Hill Book Company, New York, (1992, 4^{th} edition) pp. 699.

The thiazole moiety of the present invention may be prepared by simple modification of known procedures. See, e.g., U.S. Pat. Nos. 6,566,530; 6,608,072; 6,344,562; 6,121,455; and 5,731,442 all of which are hereby incorporated by reference. In general, the thiazole group is formed by reacting an α-bromoketone with a thioamide. In one embodiment, the functional groups that are attached to the thiazole ring may be introduced onto the α-bromoketone or thioamide before the thiazole ring is formed. However, functional groups may be introduced onto the thiazole ring after thiazole ring has been formed.

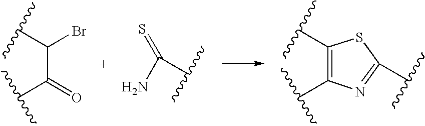

As an illustrative example for forming thiazole rings, Revesz reports in U.S. Pat. No. 6,608,072 that a thiazole ring was formed in 70% yield by simply heating the constituent α-bromoketone and thioamide in DMF for 30 min. In this preparation, 2-bromo-2-(2-fluoro-4-pyridyl)-1-(4-fluorophenyl)ethanone (2.5 g 8.0 mmol) and N-ethoxycarbonyl-piperidine-4-thiocarboxamide (2.1 g 9.6 mmol) were heated at 60° C. in DMF (4 mL) for 30 min. The reaction mixture was poured on water and extracted three times with ethyl acetate. The combined organic phases were then dried over Na₂SO₄, filtered, evaporated to dryness, and purified by SiO₂ chromatography (ethyl acetate/cyclohexane 20/80 to 100/0) to yield 4-(4-fluorophenyl)-2-(1-ethoxycarbonylpiperidin-4-yl)-5-(2-fluoro-4-pyridinyl)thiazole as an oil (2.5 g 70%).

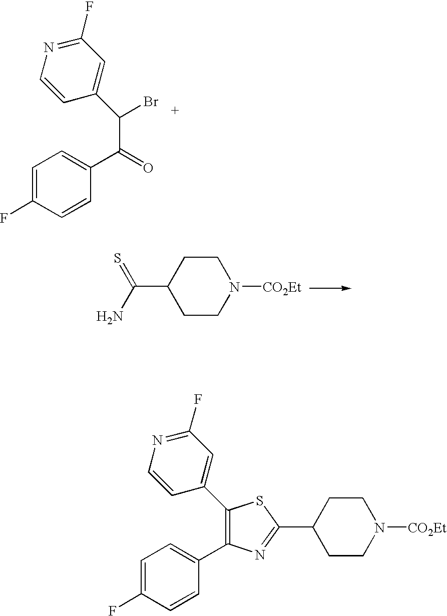

The method reported by Revesz could be used to prepare the thiazole moiety of the compounds of the invention. The R′ substituent of the thiazole group in compounds A-D of the present invention corresponds to the fluoropyridine group of the bromoketone in the Revesz example. Importantly, the nature of the R′ group does not affect the thiazole ring-forming reaction. Hence, the α-bromoketone could be selected, or prepared, wherein the fluoropyridine group of the Revesz example is replaced with H, alkyl, cycloalkyl, alkenyl, ect. In addition, the fluorophenyl group of the bromoketone in the Revesz example could be replaced with H, alkyl, cycloalkyl, alkenyl, etc.

In regards to substituent Z of compounds A-D of the present invention, substituent Z corresponds to the piperidine substituent of the thioamide in the Revesz example. Importantly, the nature of the Z substituent is not critical to the thiazole ring-forming reaction as long as it does not react with the α-bromoketone. Hence, the functional group represented by Z in compounds A-D of the invention could be installed into the thioamide moiety prior to forming the thiazole ring. The thioamide could be prepared from an amide by treatment with Lawesson's reagent. See, e.g., J. March, Advanced Organic Chemistry, McGraw Hill Book Company, New York, (1992, 4^{th} edition) pp. 893-894. Thus, a wide array of thioamides could be prepared because there are a large number of commercially available amides reported in the literature or which are easily prepared using known methods.

Alternatively, Coppola reports a method to make thiazoles in U.S. Pat. No. 6,566,530. The Coppola method involves reacting an α,β-epoxyaldehyde with thiourea to make an amino substituted thiazole in one step. As an illustrative example, Coppola reports that epoxy propanal was prepared by adjusting a solution of 800 mL water and 118 g of 30% hydrogen peroxide (1.04 mole) to a pH of 8.0-8.5 and cooling to 10° C. While maintaining a pH of 8-8.5 and a temperature of 10-20° C., 56 g (1.00 mole) of acrolein was added dropwise. The aqueous solution of the epoxy propanal was kept cold until it was used directly; however, the product may also be isolated using known procedures. Next, a solution of epoxy propanal was cooled to 0° C. With vigorous stirring, 76 g (1.00 mole) of thiourea was added in portions. After the addition is complete the water is removed under vacuum on a rotatory evaporator. The 2-amino-5-hydroxymethylthiazole product was isolated as an oil.

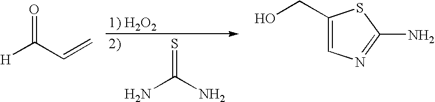

The Coppola procedure could be used to prepare compounds A-D of the invention by simply substituting an α,β-unsaturated ketone in place of the an α,β-unsaturated aldehyde. The ketone provides the methylene group to which the epoxide fragment is bonded during the Wittig reaction. See FIG. 1 (retrosynthetic analysis). The hydroxymethylene group could be reduced to the methyl group. Alternatively, the hydroxymethylene group could be oxidized to an aldehyde, ketone, ester, or carboxylic acid and optionally reacted with a nucleophile to further functionalize the thiazole ring. The OR substituent located alpha to the carbonyl group serves as a precursor to the primary bromide used in the Wittig reaction. See FIG. 1. In certain embodiments, R is a protection group which is removed and the corresponding alcohol is treated with PBr₃ to covert the hydroxyl group to a primary bromide. In a preferred embodiment, the protecting group is a benzyl or trialkylsilyl group. Importantly, all other hydroxyl or amino groups would need to be orthogonally protected before removing protecting group R.

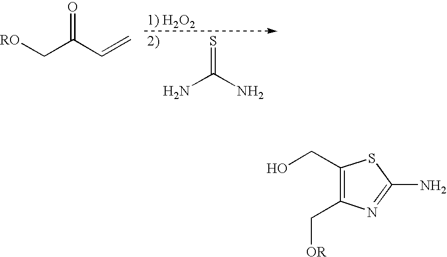

The Coppola procedure would be well-suited for preparation of compounds B and C of the invention which pertain to amino substituted thiazoles. The amino group of the thiazole ring could be reacted with an electrophile to form derivatives. For example, the amino substituted thiazole could be reacted with an alkyl or benzyl bromide to convert the primary amino group into a secondary or tertiary amino group. Alternatively, the amino substituted thiazole could be reacted with an acyl chloride to form an amide group.

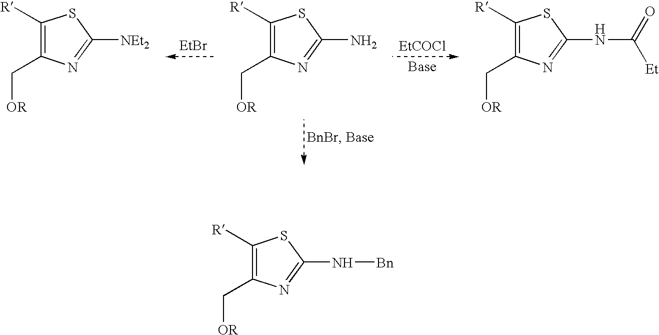

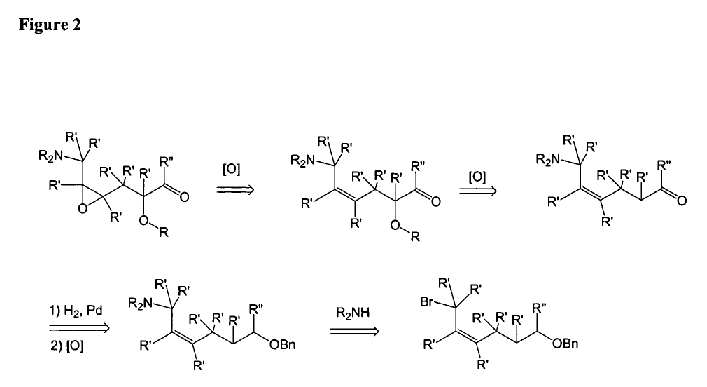

The generalized retrosynthetic route presented in FIG. 1 is amenable to compounds A and C of the invention. As depicted in FIG. 1, the thiazole moiety and the ketone or aldehyde fragment could be joined via a Wittig reaction. The epoxycarbonyl fragment could be prepared based on the retrosynthetic strategy depicted in FIG. 2. Analogous to that described above, the epoxide may be installed by epoxidation of the alkene. The oxygen atom located alpha to the carbonyl group may be installed by reaction with the Davis oxaziridine. The carbonyl group is derived from a protected hydroxyl group. The carbonyl group is formed by hydrogenation of a benzylether to remove the benzyl group followed by oxidation to the ketone or aldehyde. Finally, the amine could be installed by reacting a primary or secondary amine with an allylic leaving group. In certain embodiments, the leaving group is a halogen, mesylate, or tosylate. In a preferred embodiment, the leaving group is a bromide atom. In the event that the leaving group is a bromide, the allylic bromide can be prepared from an allylic alcohol by treatment with PBr₃.

One advantage of this synthetic route is that the epoxide substrate can easily be modified to incorporate a large variety of functional groups. For example, the intermediate ketone may be converted to its enolate and alkylated or arylated at the alpha carbonyl positions.

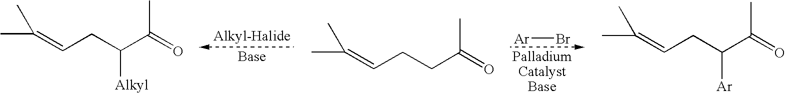

Pharmaceutical Compositions

In another aspect, the present invention provides pharmaceutically acceptable compositions which comprise a therapeutically-effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As set out above, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect, refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic a mines useful for the formation of base addition salts include ethylamine, diethylamine, ethylenediamine, ethanolamine, diethanolamine, piperazine and the like. (See, for example, Berge et al., supra) Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol, glycerol monostearate, and non-ionic surfactants; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “p arenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective a mount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular and subcutaneous doses of the compounds of this invention for a patient, when used for the indicated analgesic effects, will range from about 0.0001 to about 100 mg per kilogram of body weight per day.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical formulation (composition).

In another aspect, the present invention provides pharmaceutically acceptable compositions which comprise a therapeutically-effective amount of one or more of the subject compounds, as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin, lungs, or oral cavity; or (4) intravaginally or intravectally, for example, as a pessary, cream or foam; (5) sublingually; (6) ocularly; (7) transdermally; or (8) nasally.

The compounds according to the invention may be formulated for administration in any convenient way for use in human or veterinary medicine, by analogy with other pharmaceuticals.

The term “treatment” is intended to encompass also prophylaxis, therapy and cure.

The patient receiving this treatment is any animal in need, including primates, in particular humans, and other mammals such as equines, cattle, swine and sheep; and poultry and pets in general.

The compound of the invention can be administered as such or in admixtures with pharmaceutically acceptable carriers and can also be administered in conjunction with antimicrobial agents such as penicillins, cephalosporins, aminoglycosides and glycopeptides. Conjunctive therapy, thus includes sequential, simultaneous and separate administration of the active compound in a way that the therapeutical effects of the first administered one is not entirely disappeared when the subsequent is administered.

The addition of the active compound of the invention to animal feed is preferably accomplished by preparing an appropriate feed premix containing the active compound in an effective amount and incorporating the premix into the complete ration.

Alternatively, an intermediate concentrate or feed supplement containing the active ingredient can be blended into the feed. The way in which such feed premixes and complete rations can be prepared and administered are described in reference books (such as “Applied Animal Nutrition”, W. H. Freedman and CO., San Francisco, U.S.A., 1969 or “Livestock Feeds and Feeding” O and B books, Corvallis, Ore., U.S.A., 1977).

Combinatorial Libraries

The subject compounds readily lend themselves to preparation using the methods of combinatorial chemistry, providing access to combinatorial libraries of compounds for the screening of pharmaceutical, a grochemical or other biological or medically-related activity or material-related qualities. A combinatorial library for the purposes of the present invention is a mixture of chemically related compounds which may be screened together for a desired property; said libraries may be in solution or covalently linked to a solid support. The preparation of many related compounds in a single reaction greatly reduces and simplifies the number of screening processes which need to be carried out. Screening for the appropriate biological, pharmaceutical, agrochemical or physical property may be done by conventional methods.

Diversity in a library can be created at a variety of different levels. For instance, the substrate aryl groups used in a combinatorial approach can be diverse in terms of the core aryl moiety, e.g., a variegation in terms of the ring structure, and/or can be varied with respect to the other substituents.

A variety of techniques are available in the art for generating combinatorial libraries of small organic molecules. See, for example, Blondelle et al. (1995) Trends Anal. Chem. 14:83; the Affymax U.S. Pat. Nos. 5,359,115 and 5,362,899: the Ellman U.S. Pat. No. 5,288,514: the Still et al. PCT publication WO 94/08051; Chen et al. (1994) JACS 116:2661: Kerr et al. (1993) JACS 115:252; PCT publications WO92/10092, WO93/09668 and WO91/07087; and the Lemer et al. PCT publication WO93/20242). Accordingly, a variety of libraries on the order of about 16 to 1,000,000 or more diversomers can be synthesized and screened for a particular activity or property.

In an exemplary embodiment, a library of substituted diversomers can be synthesized using the subject reactions adapted to the techniques described in the Still et al. PCT publication WO 94/08051, e.g., being linked to a polymer bead by a hydrolyzable or photolyzable group, e.g., located at one of the positions of substrate. According to the Still et al. technique, the library is synthesized on a set of beads, each bead including a set of tags identifying the particular diversomer on that bead. In one embodiment, which is particularly suitable for discovering enzyme inhibitors, the beads can be dispersed on the surface of a permeable membrane, and the diversomers released from the beads by lysis of the bead linker. The diversomer from each bead will diffuse a cross the membrane to an assay zone, where it will interact with an enzyme assay. Detailed descriptions of a number of combinatorial methodologies are provided below.

A. Direct Characterization

A growing trend in the field of combinatorial chemistry is to exploit the sensitivity of techniques such as mass spectrometry (MS), e.g., which can be used to characterize sub-femtomolar amounts of a compound, and to directly determine the chemical constitution of a compound selected from a combinatorial library. For instance, where the library is provided on an insoluble support matrix, discrete populations of compounds can be first released from the support and characterized by MS. In other embodiments, as part of the MS sample preparation technique, such MS techniques as MALDI can be used to release a compound from the matrix, particularly where a labile bond is used originally to tether the compound to the matrix. For instance, a bead selected from a library can be irradiated in a MALDI step in order to release the diversomer from the matrix, and ionize the diversomer for MS analysis.

B) Multipin Synthesis

The libraries of the subject method can take the multipin library format. Briefly, Geysen and co-workers (Geysen et al. (1984) PNAS 81:3998-4002) introduced a method for generating compound libraries by a parallel synthesis on polyacrylic acid-grated polyethylene pins arrayed in the microtitre plate format. The Geysen technique can be used to synthesize and screen thousands of compounds per week using the multipin method, and the tethered compounds may be reused in many assays. Appropriate linker moieties can also been appended to the pins so that the compounds may be cleaved from the supports after synthesis for assessment of purity and further evaluation (c.f., Bray et al. (1990) Tetrahedron Lett 31:5811-5814; Valerio et al. (1991) Anal Biochem 197:168-177; Bray et al. (1991) Tetrahedron Lett 32:6163-6166).

C) Divide-Couple-Recombine

In yet another embodiment, a variegated library of compounds can be provided on a set of beads utilizing the strategy of divide-couple-recombine (see, e.g., Houghten (1985) PNAS 82:5131-5135; and U.S. Pat. Nos. 4,631,211; 5,440,016; 5,480,971). Briefly, as the name implies, at each synthesis step where degeneracy is introduced into the library, the beads are divided into separate groups equal to the number of different substituents to be added at a particular position in the library, the different substituents coupled in separate reactions, and the beads recombined into one pool for the next iteration.

In one embodiment, the divide-couple-recombine strategy can be carried out using an analogous approach to the so-called “tea bag” method first developed by Houghten, where compound synthesis occurs on resin sealed inside porous polypropylene bags (Houghten et al. (1986) PNAS 82:5131-5135). Substituents are coupled to the compound-bearing resins by placing the bags in appropriate reaction solutions, while all common steps such as resin washing and deprotection are performed simultaneously in one reaction vessel. At the end of the synthesis, each bag contains a single compound.

D) Combinatorial Libraries by Light-Directed, Spatially Addressable Parallel Chemical Synthesis

A scheme of combinatorial synthesis in which the identity of a compound is given by its locations on a synthesis substrate is termed a spatially-addressable synthesis. In one embodiment, the combinatorial process is carried out by controlling the addition of a chemical reagent to specific locations on a solid support (Dower et al. (1991) Annu Rep Med Chem 26:271-280; Fodor, S. P. A. (1991) Science 251:767; Pirrung et al. (1992) U.S. Pat. No. 5,143,854; Jacobs et al. (1994) Trends Biotechnol 12:19-26). The spatial resolution of photolithography affords miniaturization. This technique can be carried out through the use protection/deprotection reactions with photolabile protecting groups.

The key points of this technology are illustrated in Gallop et al. (1994) J Med Chem 37:1233-1251. A synthesis substrate is prepared for coupling through the covalent attachment of photolabile nitroveratryloxycarbonyl (NVOC) protected amino linkers or other photolabile linkers. Light is used to selectively activate a specified region of the synthesis support for coupling. Removal of the photolabile protecting groups by light (deprotection) results in activation of selected areas. After activation, the first of a set of amino acid analogs, each bearing a photolabile protecting group on the amino terminus, is exposed to the entire surface. Coupling only occurs in regions that were addressed by light in the preceding step. The reaction is stopped, the plates washed, and the substrate is again illuminated through a second mask, activating a different region for reaction with a second protected building block. The pattern of masks and the sequence of reactants define the products and their locations. Since this process utilizes photolithography techniques, the number of compounds that can be synthesized is limited only by the number of synthesis sites that can be addressed with appropriate resolution. The position of each compound is precisely known; hence, its interactions with other molecules can be directly assessed.

In a light-directed chemical synthesis, the products depend on the pattern of illumination and on the order of addition of reactants. By varying the lithographic patterns, many different sets of test compounds can be synthesized simultaneously; this characteristic leads to the generation of many different masking strategies.

E) Encoded Combinatorial Libraries

In yet another embodiment, the subject method utilizes a compound library provided with an encoded tagging system. A recent improvement in the identification of active compounds from combinatorial libraries employs chemical indexing systems using tags that uniquely encode the reaction steps a given bead has undergone and, by inference, the structure it carries. Conceptually, this approach mimics phage display libraries, where activity derives from expressed peptides, but the structures of the active peptides are deduced from the corresponding genomic DNA sequence. The first encoding of synthetic combinatorial libraries employed DNA as the code. A variety of other forms of encoding have been reported, including encoding with sequenceable bio-oligomers (e.g., oligonucleotides and peptides), and binary encoding with additional non-sequenceable tags.

1) Tagging with Sequenceable Bio-Oligomers

The principle of using oligonucleotides to encode combinatorial synthetic libraries was described in 1992 (Brenner et al. (1992) PNAS 89:5381-5383), and an example of such a library appeared the following year (Needles et al. (1993) PNAS 90:10700-10704). A combinatorial library of nominally 7⁷ (=823,543) peptides composed of all combinations of Arg, Gln, Phe, Lys, Val, D-Val and Thr (three-letter amino acid code), each of which was encoded by a specific dinucleotide (TA, TC, CT, AT, TT, CA and AC, respectively), was prepared by a series of alternating rounds of peptide and oligonucleotide synthesis on solid support. In this work, the amine linking functionality on the bead was specifically differentiated toward peptide or oligonucleotide synthesis by simultaneously preincubating the beads with reagents that generate protected OH groups for oligonucleotide synthesis and protected NH₂ groups for peptide synthesis (here, in a ratio of 1:20). When complete, the tags each consisted of 69-mers, 14 units of which carried the code. The bead-bound library was incubated with a fluorescently labeled antibody, and beads containing bound antibody that fluoresced strongly were harvested by fluorescence-activated cell sorting (FACS). The DNA tags were amplified by PCR and sequenced, and the predicted peptides were synthesized. Following such techniques, compound libraries can be derived for use in the subject method, where the oligonucleotide sequence of the tag identifies the sequential combinatorial reactions that a particular bead underwent, and therefore provides the identity of the compound on the bead.

The use of oligonucleotide tags permits exquisitely sensitive tag analysis. Even so, the method requires careful choice of orthogonal sets of protecting groups required for alternating co-synthesis of the tag and the library member. Furthermore, the chemical lability of the tag, particularly the phosphate and sugar anomeric linkages, may limit the choice of reagents and conditions that can be employed for the synthesis of non-oligomeric libraries. In preferred embodiments, the libraries employ linkers permitting selective detachment of the test compound library member for assay.

Peptides have also been employed as tagging molecules for combinatorial libraries. Two exemplary approaches are described in the art, both of which employ branched linkers to solid phase upon which coding and ligand strands are alternately elaborated. In the first approach (Kerr J M et al. (1993) J Am Chem Soc 115:2529-2531), orthogonality in synthesis is achieved by employing acid-labile protection for the coding strand and base-labile protection for the compound strand.

In an alternative approach (Nikolaiev et al. (1993) Pept Res 6:161-170), branched linkers are employed so that the coding unit and the test compound can both be attached to the same functional group on the resin. In one embodiment, a cleavable linker can be placed between the branch point and the bead so that cleavage releases a molecule containing both code and the compound (Ptek et al. (1991) Tetrahedron Lett 32:3891-3894). In another embodiment, the cleavable linker can be placed so that the test compound can be selectively separated from the bead, leaving the code behind. This last construct is particularly valuable because it permits screening of the test compound without potential interference of the coding groups. Examples in the art of independent cleavage and sequencing of peptide library members and their corresponding tags has confirmed that the tags can accurately predict the peptide structure.

2) Non-sequenceable Tagging: Binary Encoding

An alternative form of encoding the test compound library employs a set of non-sequencable electrophoric tagging molecules that are used as a binary code (Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. In the original report (Ohlmeyer et al., supra) the tags were bound to about 1% of the available amine groups of a peptide library via a photocleavable o-nitrobenzyl linker. This approach is convenient when preparing combinatorial libraries of peptide-like or other amine-containing molecules. A more versatile system has, however, been developed that permits encoding of essentially any combinatorial library. Here, the compound would be attached to the solid support via the photocleavable linker and the tag is attached through a catechol ether linker via carbene insertion into the bead matrix (Nestler et al. (1994) J Org Chem 59:4723-4724). This orthogonal attachment strategy permits the selective detachment of library members for assay in solution and subsequent decoding by ECGC after oxidative detachment of the tag sets.

Although several amide-linked libraries in the art employ binary encoding with the electrophoric tags attached to amine groups, attaching these tags directly to the bead matrix provides far greater versatility in the structures that can be prepared in encoded combinatorial libraries. Attached in this way, the tags and their linker are nearly as unreactive as the bead matrix itself. Two binary-encoded combinatorial libraries have been reported where the electrophoric tags are attached directly to the solid phase (Ohlmeyer et al. (1995) PNAS 92:6027-6031) and provide guidance for generating the subject compound library. Both libraries were constructed using an orthogonal attachment strategy in which the library member was linked to the solid support by a photolabile linker and the tags were attached through a linker cleavable only by vigorous oxidation. Because the library members can be repetitively partially photoeluted from the solid support, library members can be utilized in multiple assays. Successive photoelution also permits a very high throughput iterative screening strategy: first, multiple beads are placed in 96-well microtiter plates; second, compounds are partially detached and transferred to assay plates; third, a metal binding assay identifies the active wells; fourth, the corresponding beads are rearrayed singly into new microtiter plates; fifth, single active compounds are identified; and sixth, the structures are decoded.

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A compound represented by A: 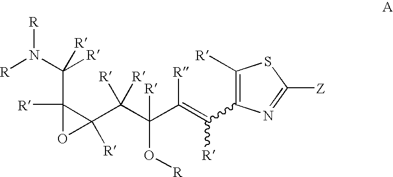
wherein
Z represents H, alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylakylamino, sulfhydryl, alkylthio, arylthio, arylakylthio, nitro, azido, alkylseleno, formyl, acyl, carboxyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₈₀;
R represents independently for each occurrence H, alkyl, cycloalkyl, alkenyl, aryl, heteroaryl, arylalkyl, trialkylsilyl, alkyldiarylsilyl, dialkylarylsilyl, triarylsilyl, formyl, acyl, alkylsulfonyl, arylsulfonyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;
R′ represents independently for each occurrence H, alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylakylamino, sulfhydryl, alkylthio, arylthio, arylakylthio, nitro, azido, alkylseleno, formyl, acyl, carboxyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₈₀;
R″ represents alkyl, cycloalkyl, alkenyl, aryl, heteroaryl, arylalkyl, formyl, acyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;
R₈₀ represents independently for each occurrence an aryl, cycloalkyl, cycloalkenyl, heterocyclyl, or polycyclyl moiety;
m is independently for each occurrence an integer in the range 0 to 8 inclusive;
the geometric configuration at an alkenyl moiety in a compound represented by A is E, Z, or a mixture thereof; and
the stereochemical configuration at a stereocenter in a compound represented by A is R, S, or a mixture thereof.

2. The compound of claim 1, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ-R₈₀.

3. The compound of claim 1, wherein R′ represents independently for each occurrence H, alkyl, cycloalkyl, alkenyl, aryl, heteroaryl, or arylalkyl.

4. The compound of claim 1, wherein R″ represents independently for each occurrence H, alkyl, cycloalkyl, alkenyl, aryl, heteroaryl, or arylalkyl.

5. The compound of claim 1, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; and R′ represents independently for each occurrence H, alkyl, cycloalkyl, alkenyl, aryl, heteroaryl, or arylalkyl.

6. The compound of claim 1, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; R′ represents independently for each occurrence H, alkyl, cycloalkyl, alkenyl, aryl, heteroaryl, or arylalkyl; and R″ represents independently for each occurrence H, alkyl, cycloalkyl, alkenyl, aryl, heteroaryl, or arylalkyl.

7. A compound represented by B: 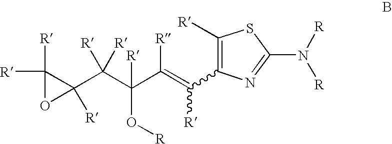
wherein
R represents independently for each occurrence H, alkyl, cycloalkyl, alkenyl, aryl, heteroaryl, arylalkyl, trialkylsilyl, alkyldiarylsilyl, dialkylarylsilyl, triarylsilyl, formyl, acyl, alkylsulfonyl, arylsulfonyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;
R′ represents independently for each occurrence H, alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylakylamino, sulfhydryl, alkylthio, arylthio, arylakylthio, nitro, azido, alkylseleno, formyl, acyl, carboxyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₈₀;
R″ represents alkyl, cycloalkyl, alkenyl, aryl, heteroaryl, arylalkyl, formyl, acyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;
R₈₀ represents independently for each occurrence an aryl, cycloalkyl, cycloalkenyl, heterocyclyl, or polycyclyl moiety;
m is independently for each occurrence an integer in the range 0 to 8 inclusive;
the geometric configuration at an alkenyl moiety in a compound represented by B is E, Z, or a mixture thereof; and
the stereochemical configuration at a stereocenter in a compound represented by B is R, S, or a mixture thereof.

8. The compound of claim 7, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀.

9. The compound of claim 7, wherein R′ represents independently for each occurrence H, alkyl, cycloalkyl, alkenyl, aryl, heteroaryl, or arylalkyl.

10. The compound of claim 7, wherein R″ represents independently for each occurrence H, alkyl, cycloalkyl, alkenyl, aryl, heteroaryl, or arylalkyl.

11. The compound of claim 7, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; and R′ represents independently for each occurrence H, alkyl, cycloalkyl, alkenyl, aryl, heteroaryl, or arylalkyl.

12. The compound of claim 7, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; R′ represents independently for each occurrence H, alkyl, cycloalkyl, alkenyl, aryl, heteroaryl, or arylalkyl; and R″ represents independently for each occurrence H, alkyl, cycloalkyl, alkenyl, aryl, heteroaryl, or arylalkyl.

13. A compound represented by C: 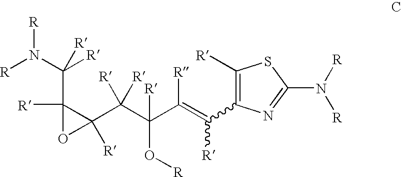
wherein
R represents independently for each occurrence H, alkyl, cycloalkyl, alkenyl, aryl, heteroaryl, arylalkyl, trialkylsilyl, alkyldiarylsilyl, dialkylarylsilyl, triarylsilyl, formyl, acyl, alkylsulfonyl, arylsulfonyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;
R′ represents independently for each occurrence H, alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylakylamino, sulfhydryl, alkylthio, arylthio, arylakylthio, nitro, azido, alkylseleno, formyl, acyl, carboxyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₈₀;
R″ represents alkyl, cycloalkyl, alkenyl, aryl, heteroaryl, arylalkyl, formyl, acyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;
R₈₀ represents independently for each occurrence an aryl, cycloalkyl, cycloalkenyl, heterocyclyl, or polycyclyl moiety;
m is independently for each occurrence an integer in the range 0 to 8 inclusive;
the geometric configuration at an alkenyl moiety in a compound represented by C is E, Z, or a mixture thereof; and
the stereochemical configuration at a stereocenter in a compound represented by C is R, S, or a mixture thereof.

14. The compound of claim 13, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ-R₈₀.

15. The compound of claim 13, wherein R′ represents independently for each occurrence H, alkyl, cycloalkyl, alkenyl, aryl, heteroaryl, or arylalkyl.

16. The compound of claim 13, wherein R″ represents independently for each occurrence H, alkyl, cycloalkyl, alkenyl, aryl, heteroaryl, or arylalkyl.

17. The compound of claim 13, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; and R′ represents independently for each occurrence H, alkyl, cycloalkyl, alkenyl, aryl, heteroaryl, or arylalkyl.

18. The compound of claim 13, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; R′ represents independently for each occurrence H, alkyl, cycloalkyl, alkenyl, aryl, heteroaryl, or arylalkyl; and R″ represents independently for each occurrence H, alkyl, cycloalkyl, alkenyl, aryl, heteroaryl, or arylalkyl.

19. A compound represented by D: 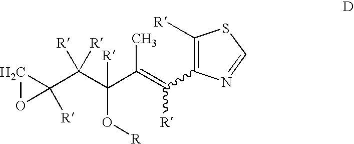
wherein
R represents H, alkyl, cycloalkyl, alkenyl, aryl, heteroaryl, arylalkyl, trialkylsilyl, alkyldiarylsilyl, dialkylarylsilyl, triarylsilyl, formyl, acyl, alkylsulfonyl, arylsulfonyl, (alkyloxy)carbonyl, (aryloxy)carbonyl, (arylalkyloxy)carbonyl, (alkylamino)carbonyl, (arylamino)carbonyl, (arylalkylamino)carbonyl, or —(CH₂)ₘ—R₈₀;
R′ represents independently for each occurrence H, alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylakylamino, sulfhydryl, alkylthio, arylthio, arylakylthio, nitro, azido, alkylseleno, formyl, acyl, carboxyl, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, arylsulfonyl, or —(CH₂)ₘ—R₈₀;
R₈₀ represents independently for each occurrence an aryl, cycloalkyl, cycloalkenyl, heterocyclyl, or polycyclyl moiety;
m is independently for each occurrence an integer in the range 0 to 8 inclusive;
the geometric configuration at an alkenyl moiety in a compound represented by D is E, Z, or a mixture thereof; and
the stereochemical configuration at a stereocenter in a compound represented by D is R, S, or a mixture thereof.

20. The compound of claim 19, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ-R₈₀.

21. The compound of claim 19, wherein R′ represents independently for each occurrence H, alkyl, cycloalkyl, alkenyl, aryl, heteroaryl, or arylalkyl.

22. The compound of claim 19, wherein R represents independently for each occurrence H, alkyl, cycloalkyl, alkenyl, aryl, heteroaryl, arylalkyl, acyl, alkylsulfonyl, arylsulfonyl, (alkyloxy)carbonyl, (aryloxy)carbonyl, (arylalkyloxy)carbonyl, or —(CH₂)ₘ—R₈₀; and R′ represents independently for each occurrence H, alkyl, cycloalkyl, alkenyl, aryl, heteroaryl, or arylalkyl.

23. The compound of claim 1, 7, 13, or 19, wherein said compound has an IC₅₀ less than 1 μM in an assay based on a mammalian GPCR or protein kinase.

24. The compound of claim 1, 7, 13, or 19, wherein said compound has an IC₅₀ less than 100 nM in an assay based on a mammalian GPCR or protein kinase.

25. The compound of claim 1, 7, 13, or 19, wherein said compound has an IC₅₀ less than 10 nM in an assay based on a mammalian GPCR or protein kinase.

26. The compound of claim 1, 7, 13, or 19, wherein said compound has an EC₅₀ less than 1 μM in an assay based on a mammalian GPCR or protein kinase.

27. The compound of claim 1, 7, 13, or 19, wherein said compound has an EC₅₀ less than 100 nM in an assay based on a mammalian GPCR or protein kinase.

28. The compound of claim 1, 7, 13, or 19, wherein said compound has an EC₅₀ less than 10 nM in an assay based on a mammalian GPCR or protein kinase.

29. The compound of claim 1, 7, 13, or 19, wherein said compound is a single stereoisomer.

30. A formulation, comprising a compound of claim 1, 7, 13, or 19; and a pharmaceutically acceptable excipient.

31. A method of modulating the activity of a GPCR or protein kinase in a mammal, comprising the step of:
administering to said mammal a therapeutically effective amount of a compound of claim 1, 7, 13, or 19.

32. The method of claim 31, wherein said mammal is a primate, equine, canine or feline.

33. The method of claim 31, wherein said mammal is a human.

34. The method of claim 31, wherein said compound is administered orally.

35. The method of claim 31, wherein said compound is administered intravenously.

36. The method of claim 31, wherein said compound is administered sublingually.

37. The method of claim 31, wherein said compound is administered ocularly.

38. The method of claim 31, wherein said compound is administered transdermally.

39. The method of claim 31, wherein said compound is administered rectally.

40. The method of claim 31, wherein said compound is administered vaginally.

41. The method of claim 31, wherein said compound is administered topically.

42. The method of claim 31, wherein said compound is administered intramuscularly.

43. The method of claim 31, wherein said compound is administered subcutaneously.

44. The method of claim 31, wherein said compound is administered buccally.

45. The method of claim 31, wherein said compound is administered nasally.

46. A method of treating a mammal suffering from addiction, anxiety, depression, sexual dysfunction, hypertension, migraine, Alzheimer's disease, obesity, emesis, psychosis, analgesia, schizophrenia, Parkinson's disease, restless leg syndrome, sleeping disorders, attention deficit hyperactivity disorder, irritable bowel syndrome, premature ejaculation, menstrual dysphoria syndrome, urinary incontinence, inflammatory pain, neuropathic pain, Lesche-Nyhane disease, Wilson's disease, Tourette's syndrome, psychiatric disorders, stroke, senile dementia, peptic ulcers, pulmonary obstruction disorders, asthma, cancer, cell proliferative disorders, fibrotic disorders, metabolic disorders, or diabetes, comprising the step of:
administering to said mammal a therapeutically effective amount of a compound of claim 1, 7, 13, or 19.

47. The method of claim 46, wherein said mammal is a primate, equine, canine or feline.

48. The method of claim 46, wherein said mammal is a human.

49. The method of claim 46, wherein said compound is administered orally.

50. The method of claim 46, wherein said compound is administered intravenously.

51. The method of claim 46, wherein said compound is administered sublingually.

52. The method of claim 46, wherein said compound is administered ocularly.

53. The method of claim 46, wherein said compound is administered transdermally.

54. The method of claim 46, wherein said compound is administered rectally.

55. The method of claim 46, wherein said compound is administered vaginally.

56. The method of claim 46, wherein said compound is administered topically.

57. The method of claim 46, wherein said compound is administered intramuscularly.

58. The method of claim 46, wherein said compound is administered subcutaneously.

59. The method of claim 46, wherein said compound is administered buccally.

60. The method of claim 46, wherein said compound is administered nasally.
